(19) United States
(12) Patent Application Publication
(10) Pub. No.: US 2019/0048422 A1
(43) Pub. Date: Feb. 14, 2019

(54) COMPOSITIONS AND METHODS FOR DETECTING AND DIAGNOSING NEOPLASIA

(71) Applicant: The Johns Hopkins University, Baltimore, MD (US)

(72) Inventors: Alicia Hulbert, Baltimore, MD (US); Alejandro Stark, Baltimore, MD (US); James G. Herman, Baltimore, MD (US); Jeff Wang, Timonium, MD (US); Malcolm Brock, Owings Mills, MD (US)

(21) Appl. No.: 15/910,629

(22) Filed: Mar. 2, 2018

Related U.S. Application Data
(63) Continuation of application No. PCT/US2016/049288, filed on Aug. 29, 2016.
(60) Provisional application No. 62/214,563, filed on Sep. 4, 2015.

(51) Int. Cl. C12Q 1/6886

(52) U.S. Cl. CPC C12Q 1/6886; C12Q 2600/118; C12Q 2600/106; C12Q 2600/154

(57) ABSTRACT

The present invention relates to the use of nucleic acid methylation and methylation profiles to detect risk of developing neoplasia and in particular, lung cancer. The invention relates to methods for identifying a methylation profile of the CDO1, SOOX17, HOXA7, HOXA9, TAC1, and ZFP42 genes from plasma and sputum samples.

### RELATED APPLICATIONS

This application is an International Patent Application that claims the benefit of priority under 35 U.S.C. § 119(e) of U.S. Provisional Application No. 62/214,563, filed on Sep. 4, 2015, which is incorporated herein by reference in its entirety.

### STATEMENT OF RIGHTS TO INVENTIONS MADE UNDER FEDERALLY SPONSORED RESEARCH

This work was supported by the following grants: Lung Cancer SPORE Grant No. CA058184, National Institutes of Health Grant Nos. CCNE U54 CA151838 and R01 CA155305, and Department of Defense Grant No. W81XWH-12-1-323. The government has certain rights in the invention.

### FIELD OF THE INVENTION

The present invention relates to the use of nucleic acid methylation and methylation profiles to detect and diagnose disease. In particular, the invention relates to methods for detecting and diagnosing lung cancer by detecting nucleic acid hypermethylation of one or more genes in one or more samples.

### BACKGROUND OF THE INVENTION

Cancer remains one of the leading causes of death in the United States. Clinically, a broad variety of medical approaches, including surgery, radiation therapy and chemotherapeutic drug therapy are currently being used in the treatment of human cancer. However, such approaches continue to be limited by an inability to predict the likelihood of metastasis and tumor recurrence and/or the most efficacious treatment regime.

Human cancer cells typically contain somatically altered nucleic acids, characterized by mutation, amplification, and/or deletion of critical genes. In addition, the nucleic acids from human cancer cells often display somatic changes in DNA methylation. However, a precise role for, and the significance of, abnormal DNA methylation in human tumorigenesis has not been well established.

DNA methylation is a chemical modification of DNA performed by enzymes called methyltransferases, in which a methyl group is added to certain cytosines of DNA. This epigenetic process plays an important role in regulating gene expression. DNA methylation also plays an important role in establishing gene expression. By turning genes off that are not needed, DNA methylation is an essential control mechanism for the normal development and functioning of organisms. Conversely, abnormal DNA methylation is one of the mechanisms underlying the changes observed with the development of many cancers. However, a precise role for, and the significance of, abnormal DNA methylation in human neoplasia has not been well established.

Loss of gene function in cancer can occur by both genetic and epigenetic mechanisms. The best-defined epigenetic alteration of cancer genes involves DNA methylation of clustered CpG dinucleotides, or CpG islands, in regulatory regions associated with the transcriptional inactivation of the affected genes (e.g., promoter regions). CpG islands are short sequences rich in the CpG dinucleotide, and can be found in the 5′ region of about half of all human genes. Methylation of cytosine within 5′ CpG islands is associated with loss of gene expression and has been seen in a number of physiological conditions, including X chromosome inactivation and genomic imprinting. Aberrant methylation of CpG islands has been detected in genetic diseases such as the fragile-X syndrome, in aging cells and in neoplasia. About half of the tumor suppressor genes which have been shown to be mutated in the germline of patients with familial cancer syndromes have also been shown to be aberrantly methylated in some proportion of sporadic cancers, including Rb, VHL, p16, hMLH1, and BRCA1. Methylation of tumor suppressor genes in cancer is usually associated with lack of gene transcription and absence of coding region mutation. Thus CpG island methylation can serve as an alternative mechanism of gene inactivation in cancer.

In normal cells, methylation occurs predominantly in regions of DNA that have few CG base repeats, while CpG islands, regions of DNA that have long repeats of CG bases, remain non-methylated. Gene promoter regions that control gene expression are often CpG island-rich. Aberrant methylation of these normally non-methylated CpG islands in the promoter region causes transcriptional inactivation or silencing of certain tumor suppressor genes involved in human cancers.

Genes that are methylated in tumor cells are strongly specific to the tissue of origin of the tumor. Molecular signatures of cancers of all types can be used to improve cancer detection, the assessment of cancer risk and response to therapy. Promoter methylation events provide some of the most promising markers for such purposes.

In general, cancer treatments have a higher rate of success if the cancer is diagnosed early, and treatment is started earlier in the disease process. A relationship between improved prognosis and stage of disease at diagnosis can be seen across a majority of cancers. Identification of the earliest changes in cells associated with cancer is thus a major focus in molecular cancer research. Diagnostic approaches based on identification of these changes in specific genes may allow implementation of early detection strategies and novel therapeutic approaches. Targeting these early changes will lead to more effective cancer treatment.

Despite advances in targeted therapy, surgery with curative intent remains the best therapeutic option for lung cancer patients with the earliest stages of disease. Ensuring in these patients that no metastatic cells have disseminated outside the area of curative resection is critical, because early spread of tumor cells is a leading cause of relapse. Despite the curative aim of early surgery, approximately 30%-40% of lung cancer patients with discrete lesions and histologically proven cancer negative lymph nodes (e.g., stage 1:T1-2N0) still die of recurrent disease. Furthermore, many of these recurrences are systemic, underscoring the probability that these patients had metastatic disease that was undetectable, and beyond the margins of surgical resection. Accordingly, there is an urgent need in the art for earlier and improved methods of detection of proliferative disease, and in particular, for improved methods of detecting and diagnosing neoplasia such as, for example, lung cancer.

### SUMMARY OF INVENTION

The present invention features methods for identifying lung cancer by detecting nucleic acid methylation of one or more genes in one or more samples, and in particular in plasma and sputum.

The present invention features methylation of one or more genes from a panel of genes (e.g., CDO1, SOOX17, HOXA7, HOXA9, TAC1, ZFP42, and the like) as a predictive biomarker for risk of developing lung cancer.

In one aspect, the invention pertains to a method of identifying a subject at risk of developing lung cancer, including the steps of obtaining one or more samples from the subject; extracting genomic DNA from the one or more samples; performing a conversion reaction on the genomic DNA in vitro to convert unmethylated cytosine to uracil by deamination; detecting nucleic acid methylation of one or more genes in the converted genomic DNA, wherein detecting nucleic acid methylation identifies a subject that is at risk of developing lung cancer.

In an embodiment, the sample is selected from the group consisting of blood, plasma, serum, saliva, sputum, and mucous.

In an embodiment, the detecting comprises a polymerase chain reaction (PCR) based technique. In an embodiment, the PCR-based technique is selected from the group consisting of methylation on beads (MOB), quantitative methylation specific PCR (QMSP), multiplex-methylation specific PCR (MMSP), and combinations thereof.

In an embodiment, the nucleic acid methylation is in the promoter region of the one or more genes.

In an embodiment, the sample is blood or sputum.

In an embodiment, the method further comprises determining a therapeutic regimen.

In an embodiment, the method further comprises imaging the subject with one or more imaging modalities. In an embodiment, the one or more imaging modalities are selected from the group comprising computed tomography (CT), ultrasound, magnetic resonance imaging (MRI), positron emission tomography (PET), optical imaging, and combinations thereof.

In an embodiment, the lung cancer is detected at an early stage.

In an embodiment, the method is performed prior to therapeutic intervention for cancer.

In an embodiment, the method is performed after therapeutic intervention for cancer.

In an embodiment, the subject has been diagnosed with cancer.

In an aspect, the invention provides a method of treating a subject having or at risk of having cancer that includes the steps of obtaining one or more samples from the subject; extracting genomic DNA from the one or more samples; performing a conversion reaction on the genomic DNA in vitro to convert unmethylated cytosine to uracil by deamination; detecting nucleic acid methylation of one or more genes in the converted genomic DNA, wherein the one or more genes are selected from the group consisting of CDO1, SOOX17, HOXA7, HOXA9, TAC1, and ZFP42, where presence of nucleic acid methylation indicates having or a risk of having lung cancer; and administering to the subject a therapeutically effective amount of a chemotherapeutic agent, thereby treating a subject having or at risk for having cancer.

In an embodiment, the nucleic acid methylation status is compared to a threshold value that distinguishes between individuals with and without cancer.

In an embodiment, the method further includes the step of comparing the nucleic acid methylation of the one or more CDO1, SOOX17, HOXA7, HOXA9, TAC1, and ZFP42 genes in the sample with a comparable sample obtained from a normal subject.

In an embodiment, the method of detecting nucleic acid methylation is performed as a high-throughput method.

In an embodiment, the methylation is detected in CpG islands associated with a promoter of one or more genes selected from the group consisting of CDO1, SOOX17, HOXA7, HOXA9, TAC1, and ZFP42.

In one aspect, the invention provides a kit for detecting cancer, that includes one or more reagents for extracting genomic DNA from the one or more samples; one or more deamination reagents converting unmethylated cytosine in the extracted genomic DNA to uracil; two or more primers for detecting nucleic acid methylation of one or more genes selected from the group consisting of CDO1, SOOX17, HOXA7, HOXA9, TAC1, and ZFP42; and, instructions for use.

In an embodiment, the two or more primers are used for quantitative methylation specific PCR (QMSP).

In one aspect, the invention provides a method of identifying a subject at risk of developing lung cancer that includes the steps of: obtaining one or more samples from the subject, where the sample may include blood, plasma, serum, saliva, sputum, and/or mucous; extracting genomic DNA from the one or more samples; performing a bisulfite conversion reaction on the genomic DNA in vitro to convert unmethylated cytosine to uracil by deamination; amplifying, by a polymerase chain reaction (PCR) based technique, the bisulfite converted genomic DNA using one or more sets of gene specific primers to detect nucleic acid methylation of one or more corresponding genes in the converted genomic DNA, wherein detecting nucleic acid methylation identifies a subject that is at risk of developing lung cancer.

In an embodiment, the method further includes the step of quantifying the amplified bisulfite converted DNA by monitoring hydrolysis of one or more molecular probes selected from the group consisting of a Taqman® probe and a Scorpion® probe.

In an embodiment, the PCR-based technique is selected from the group consisting of methylation on beads (MOB), quantitative methylation specific PCR (QMSP), multiplex-methylation specific PCR (MMSP), and combinations thereof.

In an embodiment, the nucleic acid methylation is in the promoter region of the one or more genes.

In an embodiment, the sample is blood or sputum.

In an embodiment, the method further comprises determining a therapeutic regimen.

In an embodiment, the method further comprises imaging the subject with one or more imaging modalities.

In an embodiment, the method may be performed prior to therapeutic intervention for cancer, or after therapeutic intervention for cancer.

In an aspect, the invention provides a method of treating a subject having or at risk of having cancer that includes the steps of: obtaining one or more samples from the subject; extracting genomic DNA from the one or more samples; performing a conversion reaction on the genomic DNA in vitro to convert unmethylated cytosine to uracil by deamination; amplifying, by a polymerase chain reaction (PCR) based technique, the bisulfite converted genomic DNA using one or more sets of gene specific primers to detect nucleic acid methylation of one or more corresponding genes in the converted genomic DNA, where the one or more genes are selected from the group consisting of CDO1, SOOX17, HOXA7, HOXA9, TAC1, and ZFP42 and presence of nucleic acid methylation indicates having or a risk of having lung cancer; and administering to the subject a therapeutically effective amount of a chemotherapeutic agent, thereby treating a subject having or at risk for having cancer.

In an embodiment, the amplifying step of the method further includes quantifying the amplified bisulfite converted DNA by monitoring hydrolysis of one or more molecular probes selected from the group consisting of a Taqman® probe and a Scorpion® probe.

In an embodiment, the nucleic acid methylation of the one or more genes may be compared to a threshold value that distinguishes between individuals with and without cancer.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%. An alteration may be a change in sequence relative to a reference sequence or a change in expression level, activity, or epigenetic marker (e.g., promoter methylation).

By “biologic sample” is meant any tissue, cell, fluid, or other material derived from an organism.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition. For example, the methylation level present at a promoter in a neoplasia may be compared to the level of methylation present at that promoter in a corresponding normal tissue.

“Detect” refers to identifying the presence, absence or amount of the analyte to be detected.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias may require more aggressive therapeutic regimens.

By “diagnostic” is meant any method that identifies the presence of a pathologic condition or characterizes the nature of a pathologic condition (e.g., a neoplasia). Diagnostic methods differ in their sensitivity and specificity. While a particular diagnostic method may not provide a definitive diagnosis of a condition, it suffices if the method provides a positive indication that aids in diagnosis.

The phrase “in combination with” is intended to refer to all forms of administration that provide a de-methylating agent, or the methods of the instant invention (e.g. methods of detection of methylation) together with a second agent, such as a chemotherapeutic agent, or a de-methylating agent, where the two are administered concurrently or sequentially in any order.

The term “agent” as used herein is meant to refer to a polypeptide, polynucleotide, or fragment, or analog thereof, small molecule, inhibitory RNA, or other biologically active molecule.

The term “CpG island” refers to a sequence of nucleic acid with an increased density relative to other nucleic acid regions of the dinucleotide CpG.

The term “epigenetic marker” or “epigenetic change” as used herein is meant to refer to a change in the DNA sequences or gene expression by a process or processes that do not change the DNA coding sequence itself. In an exemplary embodiment, methylation is an epigenetic marker.

By “frequency of methylation” is meant the number of times a specific promoter is methylated in a number of samples.

By “increased methylation” is meant a detectable positive change in the level, frequency, or amount of methylation. Such an increase may be by 5%, 10%, 20%, 30%, or by as much as 40%, 50%, 60%, or even by as much as 75%, 80%, 90%, or 100%. In certain embodiments, the detection of any methylation in a promoter in a subject sample is sufficient to identify the subject as having a neoplasia, a pre-cancerous lesion, or the propensity to develop a neoplasia.

The term “hypermethylation” as used herein refers to the presence of methylated alleles in one or more nucleic acids. In preferred embodiments, hypermethylation is detected using methylation specific polymerase chain reaction (MSP).

As used herein, “methylation” is meant to refer to cytosine methylation at positions C5 of cytosine, the N6 position of adenine or other types of nucleic acid methylation. Methylation can be detection by, for example, by polymerase chain reaction (PCR), including, but not limited to methylation specific PCR. Portions of the DNA regions described herein will comprise at least one potential methylation site (i.e., a cytosine) and can comprise 2, 3, 4, 5, 6, 7, 8, 9, 10, or more potential methylation sites. In preferred embodiments, methylation is detected using methylation specific polymerase chain reaction (MSP).

As used herein the term “methylation status” is meant to refer to the presence, absence and/or quantity of methylation at a particular nucleotide, or nucleotides within a portion of DNA. The methylation status of a particular DNA sequence (e.g., a DNA marker or DNA region as described herein such as, for example, CDO1, SOOX17, HOXA7, HOXA9, TAC1, ZFP42, and the like) may indicate the methylation state of every base in the sequence or can indicate the methylation state of a subset of the base pairs (e.g., cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value. In certain examples, the methylation status is determined for a particular gene such as, for example, a CDO1, SOOX17, HOXA7, HOXA9, TAC1, and ZFP42 gene. In preferred embodiments, methylation is detected using methylation specific polymerase chain reaction (MSP).

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “methylation level” is meant the number of methylated alleles of a particular gene. Methylation level may be represented as the methylation present at a target gene/reference gene×100. Any ratio that allows the skilled artisan to distinguish neoplastic tissue from normal tissue is useful in the methods of the invention. In various embodiments, a methylation ratio cutoff value is 1, 2, 3, 4, 5, 6, or 7. One skilled in the art appreciates that the cutoff value is selected to optimize both the sensitivity and the specificity of the assay. In certain embodiments, merely detecting promoter methylation of the genes CDO1, SOOX17, HOXA7, HOXA9, TAC1, and ZFP42 in a biological sample of a subject is sufficient to identify the subject as having cancer, a pre-cancerous lesion, or having a propensity to develop cancer.

By “tumor marker profile” is meant an alteration present in a subject sample relative to a reference. In one embodiment, a tumor marker profile includes promoter methylation of a gene such as, for example, CDO1, SOOX17, HOXA7, HOXA9, TAC1, and ZFP42, as well as other marker known in the art.

By “methylation profile” is meant the methylation level at two or more promoters. In one embodiment, promoter methylation of a gene such as, for example, CDO1, SOOX17, HOXA7, HOXA9, TAC1, and ZFP42 is detected.

By “sensitivity” is meant the percentage of subjects with a particular disease that are correctly detected as having the disease. For example, an assay that detects 98/100 of carcinomas has 98% sensitivity.

By “severity of neoplasia” is meant the degree of pathology. The severity of a neoplasia increases, for example, as the stage or grade of the neoplasia increases.

By “specificity” is meant the percentage of subjects without a particular disease who test negative.

The term “neoplasm” or “neoplasia” as used herein refers to inappropriately high levels of cell division, inappropriately low levels of apoptosis, or both. A neoplasm creates an unstructured mass (e.g., a tumor), which can be either benign or malignant. For example, cancer is a neoplasia. Examples of cancers include, without limitation, lung carcinoma, small cell lung carcinoma, non-small cell lung carcinoma, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The phrase “nucleic acid” as used herein refers to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The term “proliferative disorder” as used herein refers to an abnormal growth of cells. A cell proliferative disorder as described herein may be a neoplasm.

The term “gene” refers to a segment of deoxyribonucleic acid that encodes a polypeptide including the upstream and downstream regulatory sequences. Specifically, the term gene includes the promoter region upstream of the gene.

The term “promoter” or “promoter region” refers to a minimal sequence sufficient to direct transcription or to render promoter-dependent gene expression that is controllable for cell-type specific or tissue-specific gene expression, or is inducible by external signals or agents. Promoters may be located in the 5′ or 3′ regions of the gene. Promoter regions, in whole or in part, of a number of nucleic acids can be examined for sites of CpG-island methylation. In general, a promoter includes, at least, 50, 75, 100, 125, 150, 175, 200, 250, 300, 400, 500, 750, 1000, 1500, or 2000 nucleotides upstream of a given coding sequence (e.g., upstream of the coding sequence for genes such as, for example, CDO1, SOOX17, HOXA7, HOXA9, TAC1, and ZFP42 polypeptides). One of skill in the art will appreciate that a promoter location may vary outside these parameters for some genes, and also that some genes may comprise more than one promoter (e.g., multiple tissue specific promoters).

The term “sample” as used herein refers to any biological or chemical mixture for use in the method of the invention. The sample can be a biological sample. The biological samples are generally derived from a patient, preferably as a bodily fluid (such as tumor tissue, lymph node, sputum, blood, bone marrow, cerebrospinal fluid, phlegm, saliva, or urine) or cell lysate. The cell lysate can be prepared from a tissue sample (e.g. a tissue sample obtained by biopsy), for example, a tissue sample (e.g. a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In preferred embodiments, the sample is from esophageal tumor cells, tissue or origin.

By “marker” is meant any protein or polynucleotide having an alteration in methylation, expression level or activity that is associated with a disease or disorder.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

The term “stage” or “staging” as used herein is meant to refer to the extent or progression of proliferative disease, e.g. cancer, in a subject. Staging can be “clinical” and is according to the “stage classification” corresponding to the TNM classification (Rinsho, Byori, Genpatsusei Kangan Toriatsukaikiyaku (Clinical and Pathological Codes for Handling Primary Liver Cancer): 22p. Nihon Kangangaku Kenkyukai (Liver Cancer Study Group of Japan) edition (3rd revised edition), Kanehara Shuppan, 1992). Staging in certain embodiments may refer to “molecular staging” as defined by nucleic acid hypermethylation of one or more genes in one or more samples. In preferred embodiments of the invention, the “molecular stage” stage of a cancer is determined by detection of nucleic acid hypermethylation of one or more genes in a sample from the lymph nodes.

The term “subject” as used herein is meant to include vertebrates, preferably a mammal. Mammals include, but are not limited to, humans, camels, horses, goats, sheep, cows, dogs, cats, and the like.

The term “tumor” as used herein is intended to include an abnormal mass or growth of cells or tissue. A tumor can be benign or malignant.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or a combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE FIGURES

FIG. 1 shows gene promoter methylation amplification curves. Examples of gene promoter amplification curves for the each one of the three samples comparing the studied gene and B-actin are shown. On the top plot, B-actin is represented by the long dash-short dash curves showing a sigmoidal curve with positive cycle threshold (Ct) of 24 and the dotted curve represents SOX17 with a Ct of 31 in all of the three replicates respectively. On the bottom plot B-actin (long dash-short dash curve) had a Ct of 29 in the three samples and CDO1 (dash curve) had a late positive sigmoidal amplification Ct of 38 in two of the replicates and a negative amplification curve without sigmoidal shape in the other one with Ct of 43.

FIG. 2A-FIG. 2D show receiver operator classification (ROC) curves for lung cancer detection. FIG. 2A shows ROC curves comparing 3 genes with the largest areas under the curve for blood/plasma on the left while FIG. 2B shows ROC curves comparing 3 genes with the largest areas under the curve for sputum on the right. FIG. 2C and FIG. 2D shows ROC of the combined methylation status of the genes with the largest area under the curve for blood/plasma and sputum, respectively. Abbreviations: area under the curve: AUC, 95% confidence interval: 95% CI.

FIG. 3 shows receiver operator classification curves for cancer predictions. ROC curves assessing the accuracy of the predictions for lung cancer in the testing subset for blood samples on the left and sputum samples on the right.

FIGS. 4A-4D show a schematic of methylation as a cancer biomarker. DNA methylation contributes to the progression of carcinogenesis by silencing tumor genes. FIG. 4A shows where DNA methyl transferases methylate cytosine molecules. FIG. 4B shows a spatial modeling of how methylated DNA blocks transcription. FIG. 4C shows how “normal” DNA is transcribed while methylated “cancer” DNA is not.

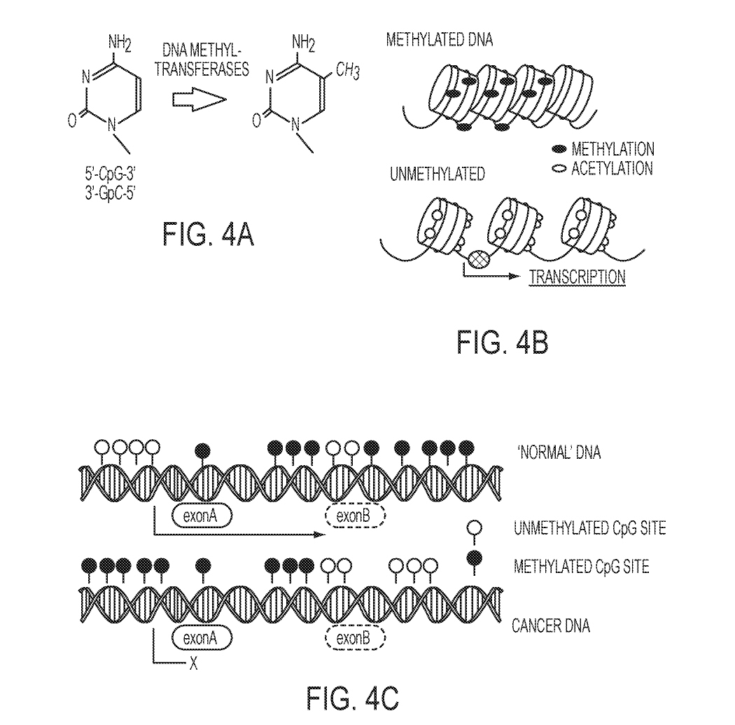

FIG. 5 shows a flowchart for identifying new DNA methylation biomarkers for detection of neoplasia such as, for example, lung cancer. DNA methylation detection in blood and sputum may be used to determine if CT (computed tomography) detected nodules are benign or malignant. Also, sputum and/or plasma may be useful for early stage diagnosis of lung cancer. Methods involve: using a case control cohort of patients with pulmonary nodules with confirmed pathology—from a chest CT (performed preoperatively) and collecting sputum and blood (also collected preoperatively). Sample processing and detection will be performed using the techniques: methylation on beads (MOB) and QMSP (Taq-man) methylation detection normalized to β-actin level. The gene panel investigated includes: CDO1, TAC1, HOXA7, HOXA9, SOX17, and ZFP42.

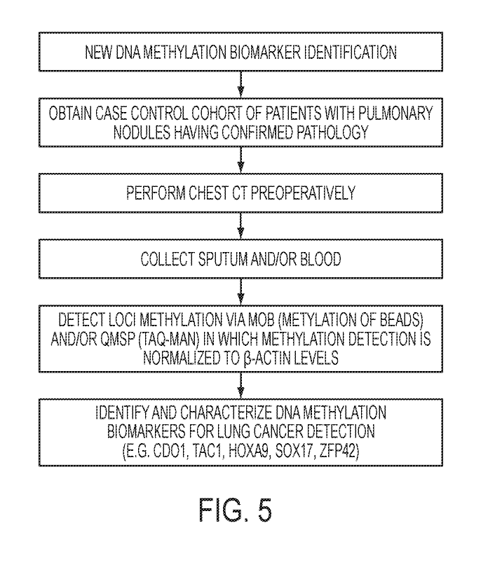

FIG. 6 shows a schematic of Integrated DNA Isolation and Bisulfite Conversion Using Silica Superparamagnetic Particles. These describe and support the Methylation on Beads (MOB) methodology.

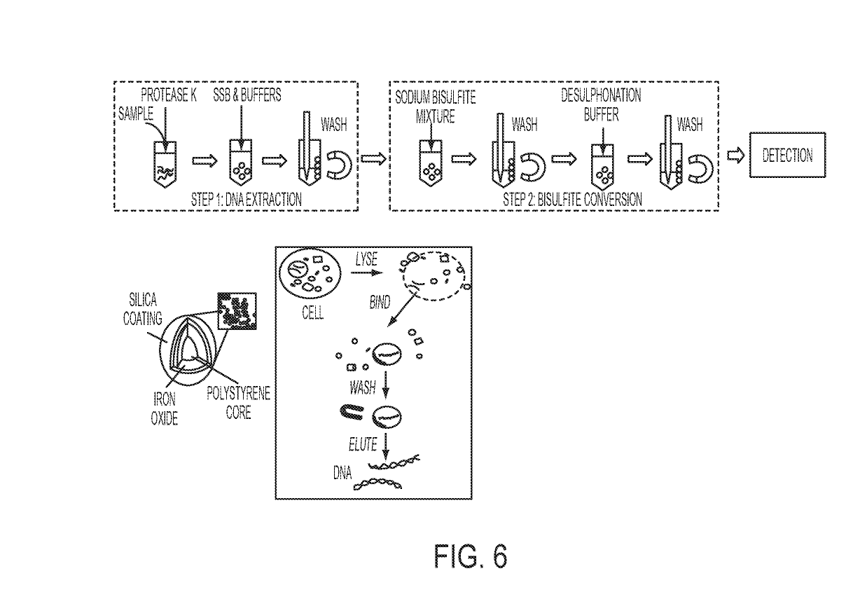

FIG. 7 shows Highly Prevalent Tumor Specific Methylation in Lung Cancer: TCGA Tumors and Normal. LUAD and LUSC, Binary Methylation, and Stage I samples are shown for each gene (HOXA9, CDO1, TAC1, SOX17, and ZFP42).

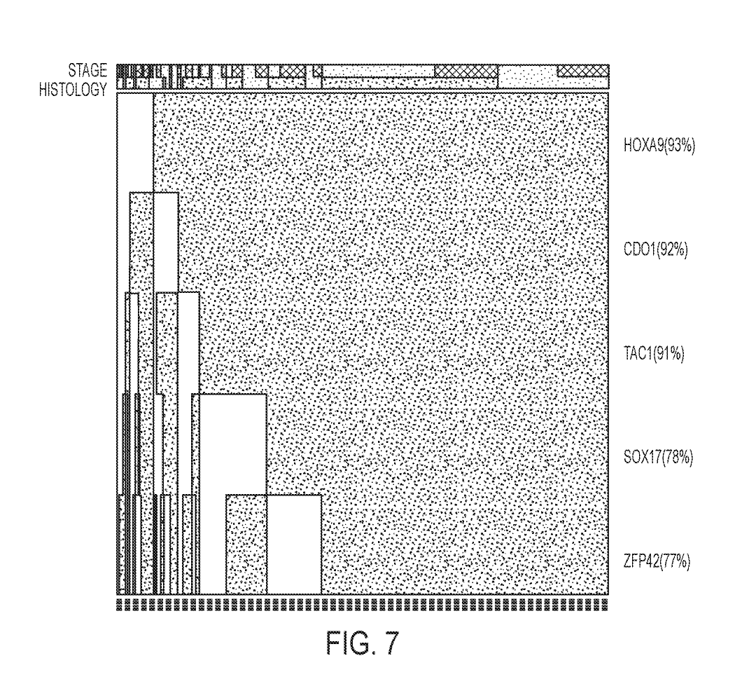

FIG. 8 shows a Table of the Study Population with patient characteristics based on age, gender, race, cancer stage, and histology.

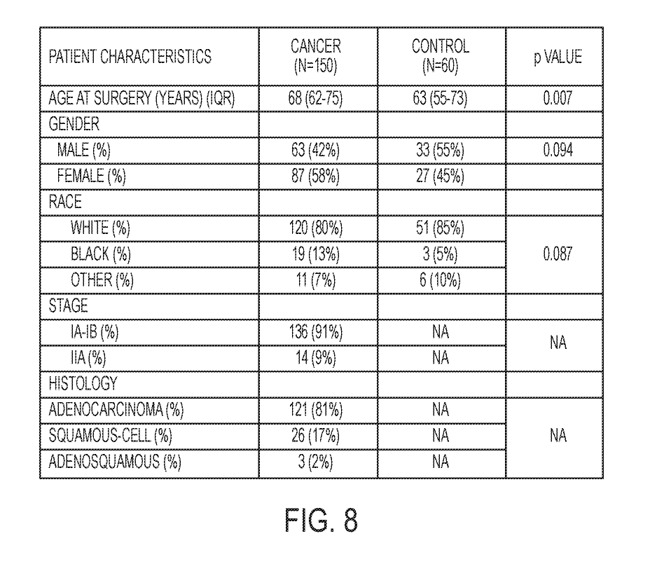

FIG. 9 shows a Table of Population Characteristics with patient characteristics based on smoking status and nodule dimensions.

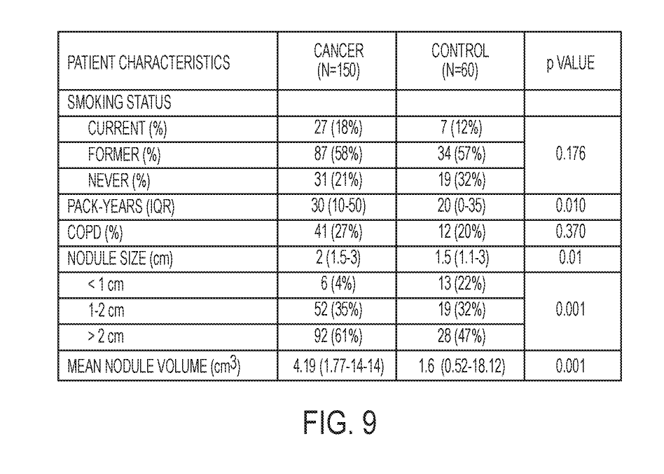

FIG. 10 shows graphs of methylation detection in plasma samples comparing B Actin to SOX17 and HOXA9.

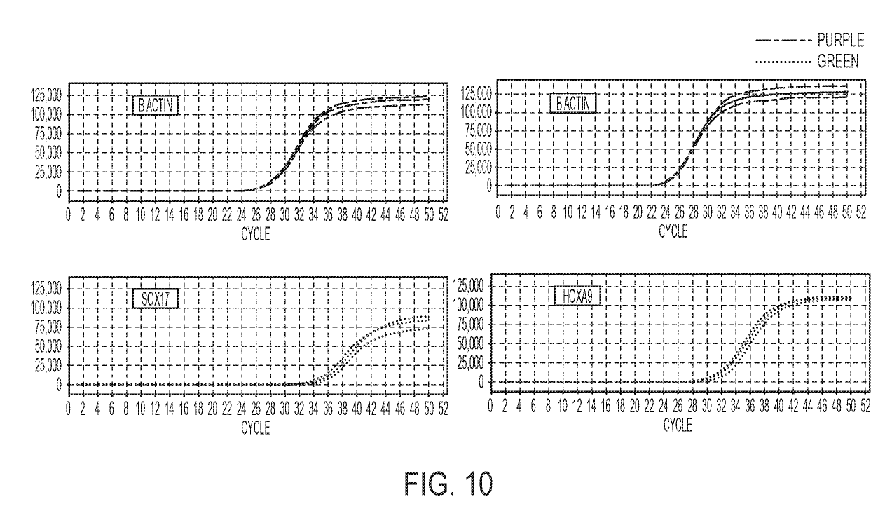

FIG. 11 shows tables for the results of methylation detection in plasma and sputum. The tables provide data for numbers of methylated genes detected. Results of methylation detection for CDO1, TAC1, HOXA7, HOXA9, SOX17, ZPF42, and the combination of CDO1, TAC1 & SOX17 is shown for blood/plasma samples. Results of methylation detection for CDO1, TAC1, HOXA7, HOXA9, SOX17, ZPF42, and the combination of TAC1, HOXA7 & SOX17 is shown for sputum samples.

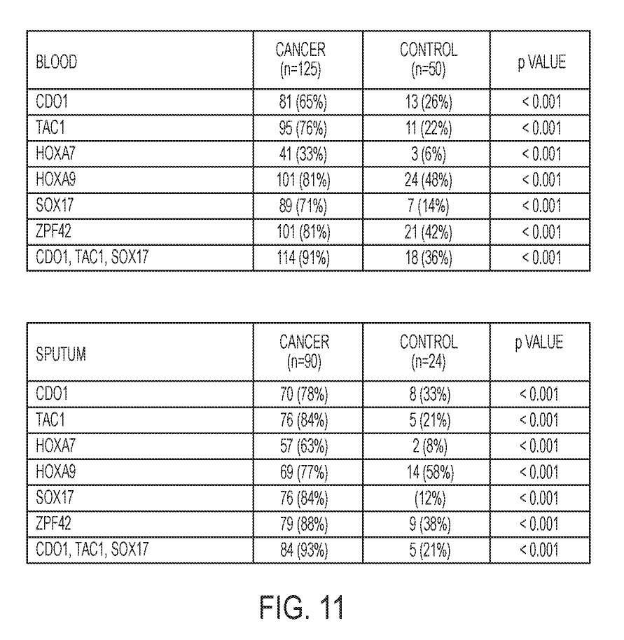

FIG. 12 shows tables for the results of methylation detection in plasma and sputum. The tables provide data for sensitivity and specificity of methylated genes detected. Results of methylation detection for CDO1, TAC1, HOXA7, HOXA9, SOX17, ZPF42, and the combination of CDO1, TAC1 & SOX17 are shown for blood/plasma samples. Results of methylation detection for CDO1, TAC1, HOXA7, HOXA9, SOX17, ZPF42, and the combination of TAC1, HOXA7 & SOX17 are shown for sputum samples.

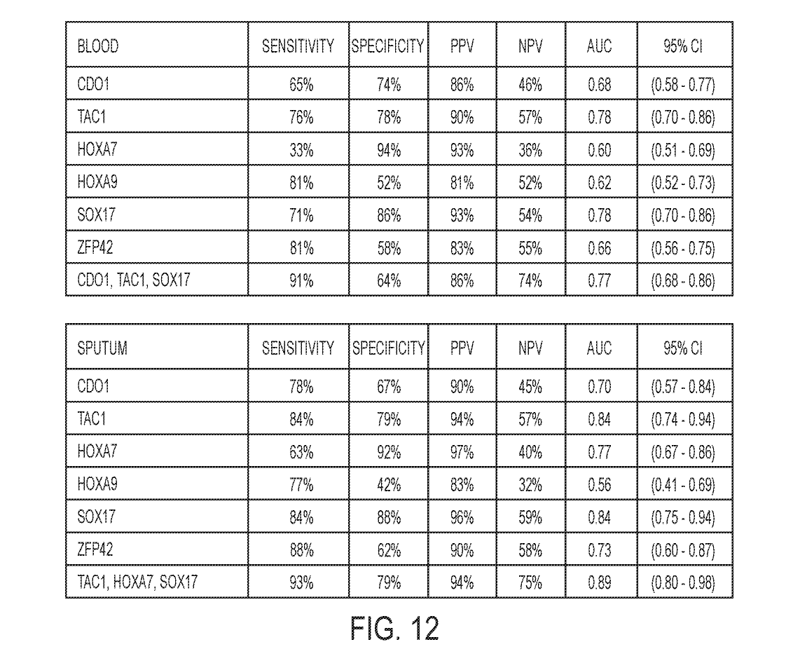

FIG. 13 shows a table of combined clinical features and methylation detection in plasma and sputum. The table depicts a blinded validation.

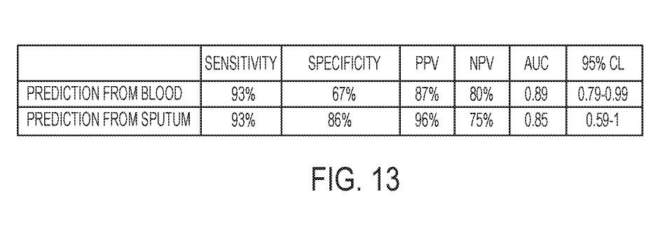

FIGS. 14A-14D show receiver operator classification (ROC) curves for lung cancer detection similar to FIG. 2. FIGS. 14A and 14B show ROC curves comparing the 3 genes with the largest areas under the curve for blood/plasma on the left and for sputum on the right (top panels). The FIGS. 14C and 14D show (bottom panels) ROC of the combined methylation status of the genes with the largest area under the curve for blood/plasma on the left and sputum on the right. Abbreviations: area under the curve: AUC, 95% confidence interval: 95% CI.

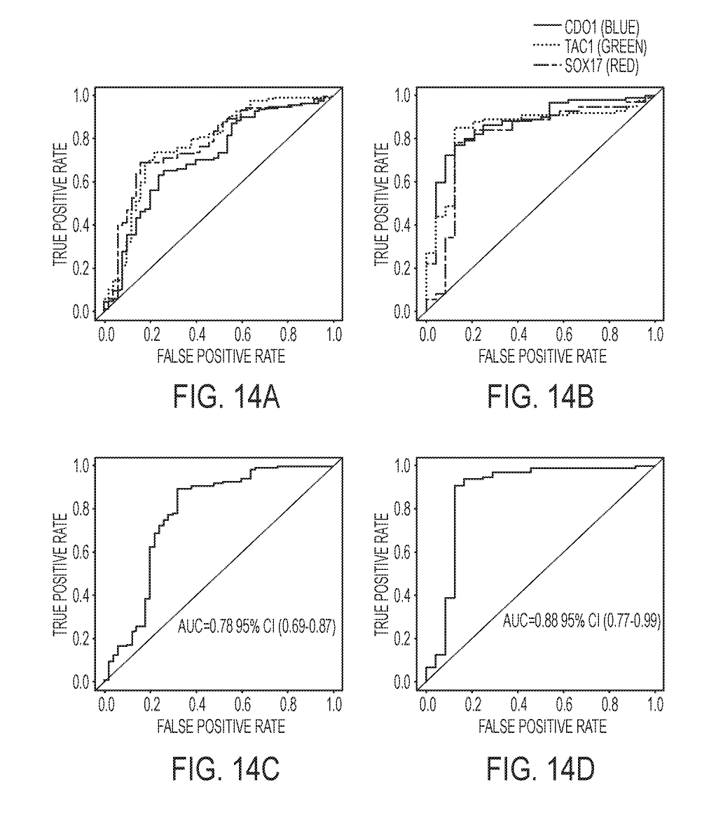

FIG. 15 shows a table of the rate of gene methylation in blood and sputum, similar to Table 2.

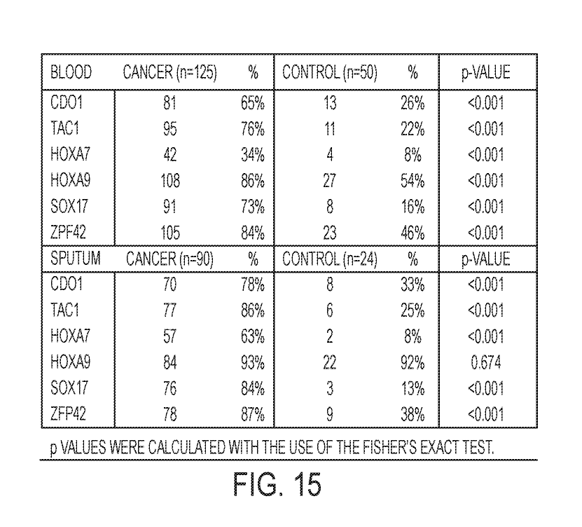

FIG. 16 shows tables depicting gene methylation sensitivity, specificity, Area Under the Curve (AUC) and association with cancer diagnosis for 1) the genes CDO1, TAC1, HOXA7, HOXA9, SOX17, ZFP42 and the combination of CDO1, TAC1, & SOX17 in blood and 2) the genes CDO1, TAC1, HOXA7, HOXA9, SOX17, ZFP42 and the combination of TAC1, SOX17, & ZFP42 in sputum.

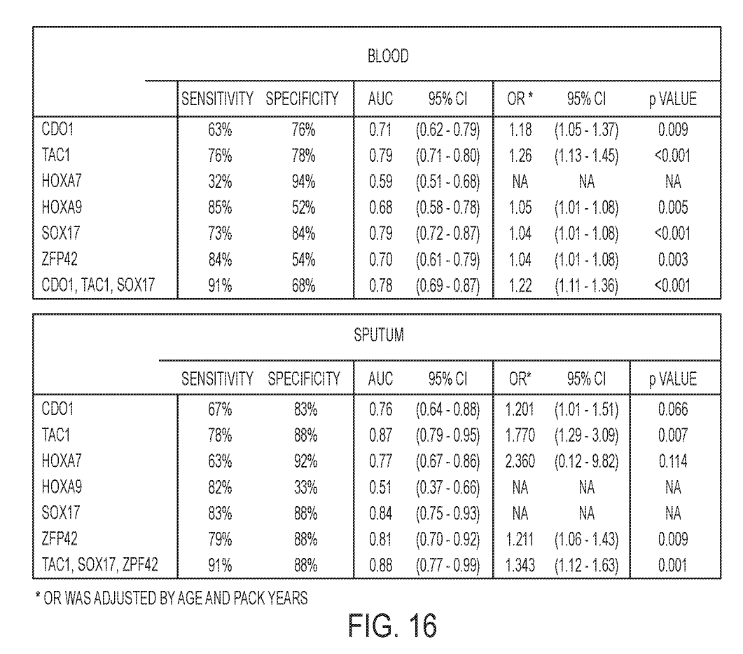

### DETAILED DESCRIPTION OF THE INVENTION

The invention features compositions and methods that are useful for identifying a subject as having or having a propensity to develop neoplasia (e.g., lung cancer). The invention is based, at least in part, upon the discovery that the methylation of certain genes (e.g., CDO1, SOOX17, HOXA7, HOXA9, TAC1, ZFP42, and the like), including promoter regions, may serve as prognostic and diagnostic markers for cellular proliferative disorders. This is the first time that promoter methylation of certain genes such as, for example, CDO1, SOOX17, HOXA7, HOXA9, TAC1, and ZFP42, in the plasma and sputum has been associated with the ability to predict risk of developing certain cancers such as, for example, lung cancer. The invention provides for an early and non-invasive method of predicting a subject's risk of developing lung cancer. DNA methylation of promoter regions leads to gene silencing in many cancers, and here DNA methylation of the CDO1, SOOX17, HOXA7, HOXA9, TAC1, and ZFP42 genes is assessed and can be correlated with clinical outcomes. Because DNA methylation does not normally occur at these loci, detection of DNA methylation simultaneously at one or more genes including, but not limited to, CDO1, SOOX17, HOXA7, HOXA9, TAC1, and ZFP42 genes (or in some instances all 6 genes) may be indicative of a subject's risk of developing lung cancer. For example, while DNA methylation can occur at the promoter region in these genes (e.g., CDO1, SOOX17, HOXA7, HOXA9, TAC1, ZFP42, and the like), methylation outside the promoter region may still be indicative that a subject is at risk of developing lung cancer. In some embodiments, DNA methylation may be detected at three genes selected from the group consisting of the CDO1, SOOX17, HOXA7, HOXA9, TAC1, and ZFP42 genes. In some embodiments, DNA methylation may be detected at two genes selected from the group consisting of the CDO1, SOOX17, HOXA7, HOXA9, TAC1, and ZFP42 genes. In some embodiments, DNA methylation may be detected at one gene selected from the group consisting of the CDO1, SOOX17, HOXA7, HOXA9, TAC1, and ZFP42 genes.

Lung cancer is the third most prevalent cancer in the United States after prostate and breast cancer, with over 63/100,000 new cases annually.^{1,2} Lung Cancer is the most deadly cancer worldwide accounting for almost 27% of all cancer-related deaths mainly because it is diagnosed at an advanced local or metastatic stage in almost 67% of cases.^{2,3} This tendency to diagnose lung cancer at a late stage results in poor survival with a 16.8% probability of survival at five-years from the time of diagnosis.² The landmark National Lung Screening Trial (NLST) demonstrated that lung cancer mortality could be reduced by 20% using low-dose computed tomography (CT) screening.⁴ Such a survival benefit comes at the price of detecting a high prevalence of indeterminate usually benign, non-calcified small pulmonary nodules at a false positive rate of 96.4%.^{4,5} This has led to the cautious adoption of CT screening, because of complications and even death related to further diagnostic and therapeutic procedures.⁶

Non-small-cell lung carcinoma (NSCLC) is any type of epithelial lung cancer other than small cell lung carcinoma (SCLC). As a class, NSCLCs are relatively insensitive to chemotherapy, compared to small cell carcinoma. When possible, they are primarily treated by surgical resection with curative intent, although chemotherapy is increasingly being used both pre-operatively (neoadjuvant chemotherapy) and post-operatively (adjuvant chemotherapy). The most common types of NSCLC are squamous cell carcinoma, large cell carcinoma, and adenocarcinoma, but there are several other types that occur less frequently, and all types can occur in unusual histologic variants and as mixed cell-type combinations. Lung cancer in never-smokers is almost universally NSCLC, with a sizeable majority being adenocarcinoma. On relatively rare occasions, malignant lung tumors are found to contain components of both SCLC and NSCLC. In these cases, the tumors should be classified as combined small cell lung carcinoma (c-SCLC), and are (usually) treated like “pure” SCLC.

Adenocarcinoma of the lung is currently the most common type of lung cancer in “never smokers” (lifelong non-smokers). Adenocarcinomas account for approximately 40% of lung cancers. Historically, adenocarcinoma was more often seen peripherally in the lungs than small cell lung cancer and squamous cell lung cancer, both of which tended to be more often centrally located. Interestingly, however, recent studies suggest that the “ratio of centrally-to-peripherally occurring” lesions may be converging toward unity for both adenocarcinoma and squamous cell carcinoma.

Squamous cell carcinoma (SCC) of the lung is more common in men than in women. It is closely correlated with a history of tobacco smoking, more so than most other types of lung cancer. According to the Nurses' Health Study, the relative risk of SCC is approximately 5.5, both among those with a previous duration of smoking of 1 to 20 years, and those with 20 to 30 years, compared to never-smokers. The relative risk increases to approximately 16 with a previous smoking duration of 30 to 40 years, and approximately 22 with more than 40 years.

Large cell lung carcinoma (LCLC) is a heterogeneous group of undifferentiated malignant neoplasms originating from transformed epithelial cells in the lung. LCLC's have typically comprised around 10% of all NSCLC in the past, although newer diagnostic techniques seem to be reducing the incidence of diagnosis of “classic” LCLC in favor of more poorly differentiated squamous cell carcinomas and adenocarcinomas. LCLC is, in effect, a “diagnosis of exclusion,” in that the tumor cells lack light microscopic characteristics that would classify the neoplasm as a small-cell carcinoma, squamous-cell carcinoma, adenocarcinoma, or other more specific histologic type of lung cancer. LCLC is differentiated from small cell lung carcinoma (SCLC) primarily by the larger size of the anaplastic cells, a higher cytoplasmic-to-nuclear size ratio, and a lack of “salt-and-pepper” chromatin.

One approach to improving the specificity of CT screening would be the use of cancer specific biomarkers. DNA-based biomarkers hold promise in that these represent cancer specific molecular changes, which can be detected in body fluids, such as sputum and plasma. In addition to mutational changes, over the past decades, a growing body of evidence has demonstrated that epigenetic gene changes, including promoter DNA methylation, are associated with the initiation and progression of various malignancies including lung cancer.⁷⁻¹³ The involvement of gene promoter methylation in carcinogenesis has led to studies focused on establishing the utility of methylation as a biomarker in screening for cancer risk, prevention, treatment, and prognosis.¹⁴⁻²⁴

Previous approaches were limited by sensitivity and specificity, which were not ideal. The extraction and processing methods used to detect DNA methylation in previous studies usually involve DNA phenol chloroform extraction and bisulfite conversion. Reduced sensitivity of the methylation assay may be related to limitations of these methods. As an alternative, Methylation-on-Beads (MOB) successfully combines these two processes into a single-tube; thereby allowing for increased throughput in detection and therefore an efficient, diagnostically sensitive methylation detection.²⁵⁻²⁷ MOB successfully combines DNA extraction with bisulfate conversion into a single process by increasing DNA detection and therefore an efficient, diagnostically sensitive methylation detection.

In addition, previous genes used for DNA methylation detection were primarily chosen from a candidate gene approach and were methylated in only a subset of lung tumors. The Cancer Genome Atlas (TCGA) project identified methylation changes across different populations and tumor types.²⁸ From the TCGA data, genes were selected with the highest methylation change in cancer such as, for example, CDO1, SOX17, HOXA7, HOXA9, TAC1 and ZFP42, as candidates for a biomarker panel.²³ An assessment of the diagnostic accuracy of the use of gene promoter methylation in sputum and plasma using MOB and QMSP in the aforementioned panel of genes as a biomarker for early detection of lung cancer in a prospective case-control study was sought.

According to the techniques herein, the risk of having all stages lung cancer may be assessed from sputum and plasma by the detection of methylation levels in a panel of genes. The techniques herein provide a high diagnostic accuracy for lung cancer. It is based on Methylation-on-Beads (MOB) and Quantitative Methylation Specific Polymerase Chain Reaction (QMSP). Epigenetic biomarkers in this application allow one to identify patients with high risk of lung cancer development, reducing further unnecessary invasive tests and increasing the chance to diagnose lung cancer at earlier stages. It is particularly useful when combined with lung cancer screening tools such as CT screening.

Prior art tests cannot provide a high degree of accuracy in identifying patients at high risk of lung cancer using the methylation status in the promoter of genes in sputum and plasma like the techniques described herein. The novelty of this method is based, at least in part, on three elements. First, it is based on the use of a unique panel of genes (e.g., CDO1, SOX17, HOXA7, HOXA9, TAC1 and ZFP42) whose methylation is highly sensitive and specific for lung cancer. Second, the techniques herein make use of Methylation-on-Beads (MOB), which is a more efficient methylation detection technique than previous technologies. The MOB method has been optimized in this patent specifically for plasma and sputum from individuals with lung cancer. This is the first time that this method has been used in clinical samples for lung cancer detection. Finally, this lung cancer risk assessment is clinically very useful to increase survival. Furthermore, this method requires a minimal amount of blood and sputum.

The lung cancer risk assessment is obtained by the detection of the methylation levels of the gene promoters from the sputum and plasma samples by using MOB and QMSP in the aforementioned unique panel of genes as biomarkers for early detection of lung cancer. MOB (methylation-on-Beads) is a process that allows DNA extraction and Bisulfite conversion in a single tube via the use of silica super magnetic beads. Both methods (MOB and QMSP) have been modified and optimized for methylation detection in body fluids for plasma and sputum in individuals with lung cancer and have never been previously used for lung cancer clinical samples. This optimization yielded a high sensitivity and specificity for detection of individuals with lung cancer especially those with stage 1, the earliest stage.

### I. Detection of Methylation

DNA methylases transfer methyl groups from the universal methyl donor S-adenosyl methionine to specific sites on the DNA. Several biological functions have been attributed to the methylated bases in DNA. The most established biological function for methylated DNA is the protection of DNA from digestion by cognate restriction enzymes. The restriction modification phenomenon has, so far, been observed only in bacteria. However, mammalian cells possess a different methylase that exclusively methylates cytosine residues that are 5′ neighbors of guanine (CpG). This modification of cytosine residues has important regulatory effects on gene expression, especially when involving CpG rich areas, known as CpG islands, located in the promoter regions of many genes.

Methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, New York, 1984). In eukaryotic cells, methylation of cytosine residues that are immediately 5′ to a guanosine, occurs predominantly in CG poor regions (Bird, A., Nature, 321:209, 1986). In contrast, CpG islands remain unmethylated in normal cells, except during X-chromosome inactivation and parental specific imprinting (Li, et al., Nature, 366:362, 1993) where methylation of 5′ regulatory regions can lead to transcriptional repression. De novo methylation of the Rb gene has been demonstrated in a small fraction of retinoblastomas (Sakai, et al., Am. J. Hum. Genet., 48:880, 1991), and recently, a more detailed analysis of the VHL gene showed aberrant methylation in a subset of sporadic renal cell carcinomas (Herman, et al., Proc. Natl. Acad. Sci., U.S.A., 91:9700, 1994). Expression of a tumor suppressor gene can also be abolished by de novo DNA methylation of a normally unmethylated CpG island (Issa, et al., Nature Genet., 7:536, 1994; Herman, et al., supra; Merlo, et al., Nature Med., 1:686, 1995; Herman, et al., Cancer Res., 56:722, 1996; Graff, et al., Cancer Res., 55:5195, 1995; Herman, et al., Cancer Res., 55:4525, 1995).

In higher order eukaryotes DNA is methylated only at cytosines located 5′ to guanosine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females. Aberrant methylation of normally unmethylated CpG islands has been described as a frequent event in immortalized and transformed cells, and has been associated with transcriptional inactivation of defined tumor suppressor genes in human cancers. Any method that is sufficient to detect methylation is a suitable for use in the methods of the invention. Any method that is sufficient to detect hypermethylation, e.g. a method that can detect methylation of nucleotides at levels as low as 0.1%, is a suitable for use in the methods of the invention. A number of different methods can be used to detect hypermethylation.

According to the techniques herein, PCR analysis is preferred, and more particularly, methylation-specific PCR analysis, for example qualitative methylation specific PCR (QMSP). Other methods that can be used include, but are not limited to, bisulfate modification to identify changes in DNA methylation of the genes including, but not limited to, CDO1, SOOX17, HOXA7, HOXA9, TAC1, and ZFP42 genes. This correlates with loss of expression. Additional methods to determine the methylation status of this gene include genomic bisulfite sequencing, MassSPEC methods of methylation detection, and those relying on methylation sensitive restriction digestion of DNA or methyl binding proteins. Other methods which examine loss of expression of the gene, for example RT-PCR approaches, or protein expression, for example immunohistochemistry or western blot analysis, might also be used to determine inactivation of genes including, but not limited to, CDO1, SOOX17, HOXA7, HOXA9, TAC1, and ZFP42 genes and thus risk of developing lung cancer.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa I, and Not I. Alternatively, chemical reagents can be used which selectively modify either the methylated or non-methylated form of CpG dinucleotide motifs.

Modified products can be detected directly, or after a further reaction which creates products which are easily distinguishable. Techniques that detect altered size and/or charge can be used to detect modified products, including but not limited to electrophoresis, chromatography, and mass spectrometry. Other techniques that are reliant on specific sequences can be used, including but not limited to hybridization, amplification, sequencing, and ligase chain reaction. Combinations of such techniques can be uses as is desired. Examples of such chemical reagents for selective modification include hydrazine and bisulfite ions. Hydrazine-modified DNA can be treated with piperidine to cleave it. Bisulfite ion-treated DNA can be treated with alkali.

Other techniques that can be used include technologies suitable for detecting DNA methylation with the use of bisulfite treatment include MSP, Mass Array, MethylLight, QAMA (quantitative analysis of methylated alleles), ERMA (enzymatic regional methylation assay), HeavyMethyl, pyrosequencing technology, MS-SNuPE, Methylquant, oligonucleotide-based microarray.

The ability to monitor the real-time progress of the PCR changes the way that PCR-based quantification of DNA and RNA may be approached. Reactions are characterized by the point in time during cycling when amplification of a PCR product is first detected rather than the amount of PCR product accumulated after a fixed number of cycles. The higher the starting copy number of the nucleic acid target, the sooner a significant increase in fluorescence is observed. An amplification plot is the plot of fluorescence signal versus cycle number. In the initial cycles of PCR, there is little change in fluorescence signal. This defines the baseline for the amplification plot. An increase in fluorescence above the baseline indicates the detection of accumulated PCR product. A fixed fluorescence threshold can be set above the baseline. The parameter C_{T} (threshold cycle) is defined as the fractional cycle number at which the fluorescence passes the fixed threshold. For example, the PCR cycle number at which fluorescence reaches a threshold value of 10 times the standard deviation of baseline emission may be used as C_{T} and it is inversely proportional to the starting amount of target cDNA. A plot of the log of initial target copy number for a set of standards versus C_{T} is a straight line. Quantification of the amount of target in unknown samples is accomplished by measuring C_{T} and using the standard curve to determine starting copy number.

The entire process of calculating C_{TS}, preparing a standard curve, and determining starting copy number for unknowns can be performed by software, for example that of the 7700 system or 7900 system of Applied Biosystems. Real-time PCR requires an instrumentation platform that consists of a thermal cycler, computer, optics for fluorescence excitation and emission collection, and data acquisition and analysis software. These machines, available from several manufacturers, differ in sample capacity (some are 96-well standard format, others process fewer samples or require specialized glass capillary tubes), method of excitation (some use lasers, others broad spectrum light sources with tunable filters), and overall sensitivity. There are also platform-specific differences in how the software processes data. Real-time PCR machines are available at core facilities or labs that have the need for high throughput quantitative analysis.

Briefly, in the Q-PCR method the number of target gene copies can be extrapolated from a standard curve equation using the absolute quantitation method. For each gene, cDNA from a positive control is first generated from RNA by the reverse transcription reaction. Using about 1 μl of this cDNA, the gene under investigation is amplified using the primers by means of a standard PCR reaction. The amount of amplicon obtained is then quantified by spectrophotometry and the number of copies calculated on the basis of the molecular weight of each individual gene amplicon. Serial dilutions of this amplicon are tested with the Q-PCR assay to generate the gene specific standard curve. Optimal standard curves are based on PCR amplification efficiency from 90 to 100% (100% meaning that the amount of template is doubled after each cycle), as demonstrated by the slope of the standard curve equation. Linear regression analysis of all standard curves should show a high correlation (R² coefficient 0.98). Genomic DNA can be similarly quantified.

When measuring transcripts of a target gene, the starting material, transcripts of a housekeeping gene are quantified as an endogenous control. Beta-actin is one of the most used nonspecific housekeeping genes. For each experimental sample, the value of both the target and the housekeeping gene are extrapolated from the respective standard curve. The target value is then divided by the endogenous reference value to obtain a normalized target value independent of the amount of starting material.

The above-described quantitative real-time PCR methodology has been adapted to perform quantitative methylation-specific PCR (QMSP) by utilizing the external primers pairs in round one (multiplex) PCR and internal primer pairs in round two (real time MSP) PCR. Thus each set of genes has one pair of external primers and two sets of three internal primers/probes (internal sets are specific for unmethylated or methylated DNA). The external primer pairs can co-amplify a cocktail of genes, each pair selectively hybridizing to a member of the panel of genes being investigated using the invention method. The method of methylation-specific PCR (QMSP) has been described in US Patent Application 20050239101, incorporated by reference in its entirety herein.

Methylation can be detected using two-stage, or “nested” PCR, for example as described in U.S. Pat. No. 7,214,485, incorporated by reference in its entirety herein. For example, two-stage, or “nested” polymerase chain reaction method is disclosed for detecting methylated DNA sequences at sufficiently high levels of sensitivity to permit cancer screening in biological fluid samples, such as e.g. sputum, obtained non-invasively.

A method for assessing the methylation status of any group of CpG sites within a CpG island, independent of the use of methylation-sensitive restriction enzymes, is described in U.S. Pat. No. 6,017,704, which is incorporated by reference in its entirety herein and described briefly as follows. This method employs primers that specific for the bisulfite reaction such that the PCR reaction itself is used to distinguish between the chemically modified methylated and unmethylated DNA, which adds an improved sensitivity of methylation detection. Unlike previous genomic sequencing methods for methylation identification which utilizes amplification primers which are specifically designed to avoid the CpG sequences, QMSP primers themselves are specifically designed to recognize CpG sites to take advantage of the differences in methylation to amplify specific products to be identified by the invention assay. The methods of QMSP include modification of DNA by sodium bisulfite or a comparable agent that converts all unmethylated but not methylated cytosines to uracil, and subsequent amplification with primers specific for methylated versus unmethylated DNA. This method of “methylation specific PCR (MSP)” requires only small amounts of DNA, is sensitive to 0.1% of methylated alleles of a given CpG island locus, and can be performed on DNA extracted from paraffin-embedded samples, for example. In addition, MSP eliminates the false positive results inherent to previous PCR-based approaches which relied on differential restriction enzyme cleavage to distinguish methylated from unmethylated DNA.

MSP provides significant advantages over previous PCR and other methods used for assaying methylation. MSP is markedly more sensitive than Southern analyses, facilitating detection of low numbers of methylated alleles and the study of DNA from small samples. MSP allows the study of paraffin-embedded materials, which could not previously be analyzed by Southern analysis. MSP also allows examination of all CpG sites, not just those within sequences recognized by methylation-sensitive restriction enzymes. This markedly increases the number of such sites which can be assessed and will allow rapid, fine mapping of methylation patterns throughout CpG rich regions. MSP also eliminates the frequent false positive results due to partial digestion of methylation-sensitive enzymes inherent in previous PCR methods for detecting methylation. Furthermore, with MSP, simultaneous detection of unmethylated and methylated products in a single sample confirms the integrity of DNA as a template for PCR and allows a semi-quantitative assessment of allele types which correlates with results of Southern analysis. Finally, the ability to validate the amplified product by differential restriction patterns is an additional advantage.

MSP may provide information similar to genomic sequencing, but can be performed with some advantages as follows. MSP is simpler and requires less time than genomic sequencing, with a typical PCR and gel analysis taking 4-6 hours. In contrast, genomic sequencing, amplification, cloning, and subsequent sequencing may take days. MSP also avoids the use of expensive sequencing reagents and the use of radioactivity. Both of these factors make MSP better suited for the analysis of large numbers of samples. The use of PCR as the step to distinguish methylated from unmethylated DNA in MSP allows for significant increase in the sensitivity of methylation detection. For example, if cloning is not used prior to genomic sequencing of the DNA, less than 10% methylated DNA in a background of unmethylated DNA cannot be seen (Myohanen, et al., supra). The use of PCR and cloning does allow sensitive detection of methylation patterns in very small amounts of DNA by genomic sequencing (Frommer, et al., Proc. Natl. Acad. Sci. USA, 89:1827, 1992; Clark, et al., Nucleic Acids Research, 22:2990, 1994). However, this means in practice that it would require sequencing analysis of 10 clones to detect 10% methylation, 100 clones to detect 1% methylation, and to reach the level of sensitivity demonstrated with MSP (1:1000) according to the techniques, one would have to sequence 1000 individual clones.

“Multiplex methylation-specific PCR” is a unique version of methylation-specific PCR. Methylation-specific PCR is described in U.S. Pat. Nos. 5,786,146; 6,200,756; 6,017,704 and 6,265,171, each of which is incorporated herein by reference in its entirety. Multiplex methylation-specific PCR utilizes MSP primers for a multiplicity of markers, for example three or more different markers, in a two-stage nested PCR amplification reaction. The primers used in the first PCR reaction are selected to amplify a larger portion of the target sequence than the primers of the second PCR reaction. The primers used in the first PCR reaction are referred to herein as “external primers” or “DNA primers” and the primers used in the second PCR reaction are referred to herein as “MSP primers.” Two sets of primers (i.e., methylated and unmethylated for each of the markers targeted in the reaction) are used as the MSP primers. In addition in multiplex methylation-specific PCR, as described herein, a small amount (i.e., 1 μl) of a 1:10 to about 10⁶ dilution of the reaction product of the first “external” PCR reaction is used in the second “internal” MSP PCR reaction.

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers of the invention are designed to be “substantially” complementary to each strand of the oligonucleotide to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with a 5′ and 3′ oligonucleotide to hybridize therewith and permit amplification of CpG containing nucleic acid sequence.

Primers of the invention are employed in the amplification process, which is an enzymatic chain reaction that produces exponentially increasing quantities of target locus relative to the number of reaction steps involved (e.g., polymerase chain reaction or PCR). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers used in invention methods may be prepared using any suitable method, such as conventional phosphotriester and phosphodiester methods or automated embodiments thereof. In one such automated embodiment, diethylphos-phoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

In certain preferred embodiments, methylation of genes including, but not limited to, CDO1, SOOX17, HOXA7, HOXA9, TAC1, and ZFP42 genes may be determined by real-time MSP using molecular beacons. The method consists in certain embodiments of using a gene for normalization, e.g. ACTB.

The primers used in the invention for amplification of the CpG-containing nucleic acid in the specimen, after bisulfite modification, specifically distinguish between untreated or unmodified DNA, methylated, and non-methylated DNA. QMSP primers for the non-methylated DNA preferably have a T in the 3′ CG pair to distinguish it from the C retained in methylated DNA, and the complement is designed for the antisense primer. MSP primers usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention embrace oligonucleotides of sufficient length and appropriate sequence so as to provide specific initiation of polymerization on a significant number of nucleic acids in the polymorphic locus. Where the nucleic acid sequence of interest contains two strands, it is necessary to separate the strands of the nucleic acid before it can be used as a template for the amplification process. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° C. to 105° C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

As described herein, any nucleic acid specimen, in purified or nonpurified form, can be utilized as the starting nucleic acid or acids, provided it contains, or is suspected of containing, the specific nucleic acid sequence containing the target locus (e.g., CpG).

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. Preferably, a molar excess (for genomic nucleic acid, usually about 10⁸:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90° C.-100° C. from about 1 to 10 minutes, preferably from 1 to 4 minutes. After this heating period, the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Most conveniently the reaction occurs at room temperature.

In certain preferred embodiments, the agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Preferably, the method of amplifying is by PCR, as described herein and as is commonly used by those of ordinary skill in the art. Alternative methods of amplification have been described and can also be employed as long as the methylated and non-methylated loci amplified by PCR using the primers of the invention are similarly amplified by the alternative means.

The amplified products are preferably identified as methylated or non-methylated by sequencing. Sequences amplified by the methods of the invention can be further evaluated, detected, cloned, sequenced, and the like, either in solution or after binding to a solid support, by any method usually applied to the detection of a specific DNA sequence such as PCR, oligomer restriction, allele-specific oligonucleotide (ASO) probe analysis, oligonucleotide ligation assays (OLAs), and the like.

Optionally, the methylation pattern of the nucleic acid can be confirmed by restriction enzyme digestion and Southern blot analysis. Examples of methylation sensitive restriction endonucleases which can be used to detect 5′CpG methylation include SmaI, SacII, EagI, MspI, HpaII, BstUI and BssHII, for example.

The invention provides a method for detecting a cell having a hypermethylated CpG island or a cell proliferative disorder associated with hypermethylated CpG in a tissue or biological fluid of a subject, comprising contacting a target cellular component suspected of expressing a gene having a methylated CpG or having a CpG-associated disorder, with an agent which binds to the component. The target cell component can be nucleic acid, such as DNA or RNA, or protein. When the component is nucleic acid, the reagent is a nucleic acid probe or PCR primer. When the cell component is protein, the reagent is an antibody probe. The probes can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the antibody, or will be able to ascertain such, using routine experimentation.

Actively transcribed genes generally contain fewer methylated CGs than the average number in DNA. Hypermethylation can also be detected by restriction endonuclease treatment and Southern blot analysis. Therefore, in certain preferred embodiments, when the cellular component detected is DNA, restriction endonuclease analysis is preferable to detect hypermethylation of the promoter for example. Any restriction endonuclease that includes CG as part of its recognition site and that is inhibited when the C is methylated can be utilized. In certain preferred examples, the methylation sensitive restriction endonuclease is BssHII, MspI, or HpaII, used alone or in combination. Other methylation sensitive restriction endonucleases will be known to those of skill in the art.

The present invention provides methods for detecting cells, preferably cancer cells, with DNA methylation in genes including, but not limited to, CDO1, SOOX17, HOXA7, HOXA9, TAC1, and ZFP42.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation. (See Tost, J. et al. Analysis and accurate quantification of CpG methylation by MALDI mass spectrometry. Nuc Acid Res, 2003, 31, 9)

One form of chromatography, high performance liquid chromatography, is used to separate components of a mixture based on a variety of chemical interactions between a substance being analyzed and a chromatography column. DNA is first treated with sodium bisulfite, which converts an unmethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences. (See Deng, D. et al. Simultaneous detection of CpG methylation and single nucleotide polymorphism by denaturing high performance liquid chromatography. 2002 Nuc Acid Res, 30, 3.)

Hybridization is a technique for detecting specific nucleic acid sequences that is based on the annealing of two complementary nucleic acid strands to form a double-stranded molecule. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil or sequence containing cytosine, representing unmethylated and methylated DNA, respectively. Computer-based microarray technology can determine which oligonucleotides hybridize with the DNA sequence and one can deduce the methylation status of the DNA.

An additional method of determining the results after sodium bisulfite treatment would be to sequence the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a pyrogram. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR) (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction enconuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TAQMAN® system and Molecular Beacon system which have separate probes labeled with a fluorophore and a fluorescence quencher. In the SCORPION® system the labeled probe in the form of a hairpin structure is linked to the primer.

DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

The number of genes whose silencing is tested and/or detected can vary: one, two, three, four, five, or more genes can be tested and/or detected. In some examples, methylation of at least one gene is detected. In other examples, methylation of at least two genes is detected. However, methylation of any number of genes may be detected, using the methods as described herein.

For purposes of the invention, an antibody or nucleic acid probe specific for a gene or gene product may be used to detect the presence of methylation either by detecting the level of polypeptide (using antibody) or methylation of the polynucleotide (using nucleic acid probe) in biological fluids or tissues. For antibody-based detection, the level of the polypeptide is compared with the level of polypeptide found in a corresponding “normal” tissue. Oligonucleotide primers based on any coding sequence region of the promoter in gene selected from genes involved in tumor suppression, nucleic acid repair, apoptosis, anti-proliferation, ras signaling, adhesion, differentiation, development, and cell cycle regulation.

In particular embodiments, oligonucleotide primers are based on the coding sequence region of the promoter in genes including, but not limited to, CDO1, SOOX17, HOXA7, HOXA9, TAC1, and ZFP42 genes, and are useful for amplifying DNA, for example by PCR. These genes are merely listed as examples and are not meant to be limiting.

Any specimen containing a detectable amount of polynucleotide or antigen can be used. Preferably the subject is human. Using the methods of the invention, expression of genes including, but not limited to, CDO1, SOOX17, HOXA7, HOXA9, TAC1, and ZFP42 genes can be identified in a cell and the appropriate course of treatment can be employed.

In certain preferred embodiments of the invention the genes can be detected in panels consisting of the following: (1) genes involved in tumor suppression and cell adhesion; (2) genes involved in cell cycle regulation and adhesion; (3) genes involved in tumor suppression and cell cycle regulation; (4) genes involved in ras signaling and cell cycle control.

Using the methods of the invention, expression of any gene, such as genes involved in tumor suppression, nucleic acid repair, apoptosis, anti-proliferation, ras signaling, adhesion, differentiation, development, and cell cycle regulation, can be identified in a cell and the appropriate course of treatment can be employed (e.g., sense gene therapy or drug therapy). The expression pattern of the gene may vary with the stage of malignancy in the lung, therefore, a sample such as blood/plasma or sputum can be screened with a panel of gene or gene product specific reagents (i.e., nucleic acid probes or antibodies) to detect gene expression and then diagnose the stage of malignancy of the lung tumor.

Any of the methods as described herein can be used in high throughput analysis of DNA methylation. For example, U.S. Pat. No. 7,144,701, incorporated by reference in its entirety herein, describes differential methylation hybridization (DMH) for a high-throughput analysis of DNA methylation.

### II. Methods

As described herein, the present invention features methods for identifying a subject that will respond to one or more microtubule-directed therapies. In preferred embodiments, the methods comprise detecting nucleic acid methylation of the, for example, CDO1, SOOX17, HOXA7, HOXA9, TAC1, and ZFP42 genes in one or more samples, wherein detecting nucleic acid methylation identifies a subject that will respond to one or more microtubule-directed therapies.

In preferred embodiments, the subject has been screened for cancer, for example lung cancer. Any sample that has methylation of genes including, but not limited to, CDO1, SOOX17, HOXA7, HOXA9, TAC1, and ZFP42 may be predicted to be at risk of developing lung cancer.

The methods of the invention can be used to predict risk of developing lung cancer in a subject. In preferred embodiments, the method comprises detecting nucleic acid methylation of genes including, but not limited to, CDO1, SOOX17, HOXA7, HOXA9, TAC1, and ZFP42 in one or more samples, and wherein detecting nucleic acid methylation identifies risk of developing cancer in a subject.

The methods described herein may be used to determine a course of treatment for a subject. These methods comprise extracting nucleic acid from one or more cell or tissue samples, detecting nucleic acid methylation of genes including, but not limited to, CDO1, SOOX17, HOXA7, HOXA9, TAC1, and ZFP42 in the sample; and identifying the nucleic acid methylation state of the CDO1, SOOX17, HOXA7, HOXA9, TAC1, and ZFP42 genes, wherein nucleic acid methylation of one or more CDO1, SOOX17, HOXA7, HOXA9, TAC1, and ZFP42 genes indicates the subject is at risk of developing lung cancer.

The samples, in certain embodiments, can be from one or more of blood, blood plasma, serum, cells, a cellular extract, a cellular aspirate, tissues, sputum, saliva, mucous, lung lavage, lung fluid, and/or other bodily fluids.

As described herein, in certain preferred examples the genes comprise one or more CpG islands in the promoter regions. Accordingly, any gene that contains one or more CpG island in the promoter region is suitable for use in the methods of the invention; however in certain preferred examples, the one or more genes may be selected from any of the genes described in the application herein, e.g. CDO1, SOOX17, HOXA7, HOXA9, TAC1, and ZFP42.

The methods of the invention as described herein are used in certain exemplary embodiments to identify lung cancer by detecting hypermethylation of one or more genes in one or more samples. In this way, the detection of nucleic acid hypermethylation identifies early stage lung cancer.

In mammals, conditions associated with aberrant methylation of genes that can be detected or monitored include, but are not limited to, metastases associated with carcinomas and sarcomas of all kinds, including one or more specific types of cancer, e.g., a lung cancer, breast cancer, an alimentary or gastrointestinal tract cancer such as colon, esophageal and pancreatic cancer, a liver cancer, a skin cancer, an ovarian cancer, an endometrial cancer, a prostate cancer, a lymphoma, hematopoietic tumors, such as a leukemia, a kidney cancer, a bronchial cancer, a muscle cancer, a bone cancer, a bladder cancer or a brain cancer, such as astrocytoma, anaplastic astrocytoma, glioblastoma, medulloblastoma, and neuroblastoma and their metastases. Suitable pre-malignant lesions to be detected or monitored using the invention include, but are not limited to, lobular carcinoma in situ and ductal carcinoma in situ.

The invention methods can be used to assay the DNA of any mammalian subject, including, but not limited to, humans, pet (e.g., dogs, cats, ferrets) and farm animals (meat and dairy).

The invention features in certain aspects a method for identifying lung cancer in a subject comprising detecting nucleic acid hypermethylation of one or more genes in one or more samples, wherein detecting nucleic acid hypermethylation identifies cancer. The term “hypermethylation” as used herein refers to the presence of methylated alleles in one or more nucleic acids. In preferred embodiments, hypermethylation is detected using quantitative methylation specific polymerase chain reaction (QMSP). In some embodiments, QMSP can be combined with methylation on beads (MOB).

The samples, in certain embodiments, can be from plasma, serum, bone marrow, blood, saliva, sputum, mucous, lung lavage, and/or any combination thereof. Thus, the invention can be used to identify cancer (e.g., lung cancer) in a subject comprising detecting nucleic acid hypermethylation of one or more genes in blood/plasma and/or sputum, wherein detecting nucleic acid hypermethylation identifies risk of lung cancer. In certain preferred embodiments, detection of hypermethylation in the blood/plasma and/or sputum indicates an early recurring disease.

In other aspects, the invention features a method for identifying lung cancer in a subject comprising detecting nucleic acid hypermethylation of one or more genes in blood/plasma and/or sputum, wherein the genes are selected from the group consisting of: genes involved in tumor suppression, DNA repair, apoptosis, anti-proliferation, ras signaling, adhesion, differentiation, development, and cell cycle regulation, in one or more cells or tissues, wherein detecting nucleic acid hypermethylation identifies risk of lung cancer.

In other examples, the invention as described herein features a method for identifying risk of lung cancer in a subject comprising detecting nucleic acid hypermethylation of at least one or more genes in a sample comprising tumor and lymph nodes, where the sample genes are selected from genes involved in tumor suppression, nucleic acid repair, apoptosis, anti-proliferation, ras signaling, adhesion, differentiation, development, and cell cycle regulation, in one or more cells or tissues, and where detecting nucleic acid methylation identifies micrometastases.

In practice, the method for detecting or diagnosing a proliferative disease in a subject comprises, in certain embodiments, extracting nucleic acid from one or more blood/plasma and/or sputum samples, detecting nucleic acid hypermethylation of one or more genes in the sample; and identifying the nucleic acid hypermethylation state of one or more genes, wherein nucleic acid hypermethylation of genes indicates a proliferative disease. In preferred examples, the proliferative disease is cancer (e.g., lung cancer).

As described herein, in certain preferred examples, the one or more genes comprise one or more CpG islands in the promoter regions. Accordingly, any gene that contains one or more CpG island in the promoter region is suitable for use in the methods of the invention; however in certain preferred examples, the one or more genes may be selected from any genes including, but not limited to, CDO1, SOOX17, HOXA7, HOXA9, TAC1, and ZFP42.

In certain embodiments, hypermethylation of at least one of the genes is detected. In other certain embodiments, hypermethylation of one or more of the genes is detected.

The detection of hypermethylation as described in these methods can be used to detect or diagnose a proliferative disease. The detection of hypermethylation as described in these methods can be used after surgery or therapy to treat a proliferative disease. The detection of methylation as described in these methods can be used to predict the recurrence of a proliferative disease. The detection of methylation as described in these methods can be used to stage a proliferative disease. The detection of methylation as described in these methods can be used to determine a course of treatment for a subject. These embodiments are discussed in further detail herein.

### Methods of Treatment

The invention as described herein may be used to treat a subject having or at risk for having cancer (e.g., lung cancer). Accordingly, the method comprises identifying nucleic acid methylation of one or more genes including, but not limited to, CDO1, SOOX17, HOXA7, HOXA9, TAC1, and ZFP42, and administering to the subject a therapeutically effective amount of a demethylating agent, thereby treating a subject having or at risk for having cancer.

The method can be used in combination with one or more microtubule-directed therapies as described herein.

The method can be used in combination with one or more chemotherapeutic agents. Anti-cancer drugs that may be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; erlotinib; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gefitinib; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; navelbine; nivolumab; nocodazole; nogalamycin; ormaplatin; oxisuran; paclitaxel; pegaspargase; peliomycin; pemetrexed; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

### Demethylating Agents

In certain embodiments, the invention features methods of identifying an agent that de-methylates methylated nucleic acids comprising identifying one or more cell or tissue samples with methylated nucleic acid, extracting the methylated nucleic acid, contacting the nucleic acid with one or more nucleic acid de-methylating candidate agents and a control agent, and identifying the nucleic acid methylation state, wherein nucleic acid de-methylation of genes in the sample by the candidate agent compared to the control indicates a demethylating agent, thereby identifying an agent that de-methylates methylated nucleic acid.

Demethylating agents include, but are not limited to, 5-aza-2′-deoxycytidine, 5-aza-cytidine, Zebularine, procaine, and L-ethionine.

Another way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Preferably the specific polynucleotide will be one which the patient has been tested for and been found to carry a silenced version.

### III. Samples

Samples for use in the methods of the invention include cells or tissues obtained from blood, blood plasma, serum, cells, a cellular extract, a cellular aspirate, lung lavage, sputum, saliva, mucous, urine, sweat, tears, and/or any bodily fluid. Tumor DNA can be found in various body fluids and these fluids can potentially serve as diagnostic material.

Any nucleic acid specimen, in purified or nonpurified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as contained in whole human DNA.

The nucleic acid-containing sample or specimen used for detection of methylated CpG may be extracted by a variety of techniques such as that described by Maniatis, et al. (Molecular Cloning: A Laboratory Manual, Cold Spring Harbor, N.Y., pp 280, 281, 1982).

If the extracted sample is impure (e.g., plasma, serum, stool, ejaculate, sputum, saliva, ductal cells, nipple aspiration fluid, ductal lavage fluid, cerebrospinal fluid or blood or a sample embedded in paraffin), it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Preferably, the method of amplifying is by PCR, as described herein and as is commonly used by those of ordinary skill in the art. However, alternative methods of amplification have been described and can also be employed. PCR techniques and many variations of PCR are known. Basic PCR techniques are described by Saiki et al. (1988 Science 239:487-491) and by U.S. Pat. Nos. 4,683,195; 4,683,202; and 4,800,159, each of which is incorporated herein by reference.

The conditions generally required for PCR include temperature, salt, cation, pH and related conditions needed for efficient copying of the master-cut fragment. PCR conditions include repeated cycles of heat denaturation (i.e. heating to at least about 95° C.) and incubation at a temperature permitting primer: adaptor hybridization and copying of the master-cut DNA fragment by the amplification enzyme. Heat stable amplification enzymes like the pwo, Thermus aquaticus or Thermococcus litoralis DNA polymerases which eliminate the need to add enzyme after each denaturation cycle, are commercially available. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Such amplification enzymes include pwo, Escherichia coli DNA polymerase I, Klenow fragment of E. coli polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Q.beta. replicase. Preferred amplification enzymes are the pwo and Taq polymerases. The pwo enzyme is especially preferred because of its fidelity in replicating DNA.

Once amplified, the nucleic acid can be attached to a solid support, such as a membrane, and can be hybridized with any probe of interest, to detect any nucleic acid sequence. Several membranes are known to one of skill in the art for the adhesion of nucleic acid sequences. Specific non-limiting examples of these membranes include nitrocellulose (NITROPURE) or other membranes used in for detection of gene expression such as polyvinylchloride, diazotized paper and other commercially available membranes such as GENESCREEN, ZETAPROBE. (Biorad), and NYTRAN. Methods for attaching nucleic acids to these membranes are well known to one of skill in the art. Alternatively, screening can be done in a liquid phase.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

### IV. Kits

The methods of the invention are ideally suited for the preparation of kits.

The invention features kits for identifying the nucleic acid methylation state of genes including, but not limited to, CDO1, SOOX17, HOXA7, HOXA9, TAC1, and ZFP42 genes comprising gene specific primers for use in polymerase chain reaction (PCR), and instructions for use.

The invention also features kits for detecting the risk of a subject developing lung cancer by detecting nucleic acid methylation of genes including, but not limited to, CDO1, SOOX17, HOXA7, HOXA9, TAC1, and ZFP42, the kit comprising gene specific primers for use in polymerase chain reaction (PCR), and instructions for use. As described above, the PCR, in particularly preferred examples, is quantitative methylation specific PCR (QMSP). In some embodiments, QMSP is combined with methylation on beads (MOB).

In certain embodiments, any gene comprising one or more CpG islands in the promoter region can be detected using the kits of the invention. In certain preferred examples, the gene is one or more of CDO1, SOOX17, HOXA7, HOXA9, TAC1, and ZFP42.

The invention features kits for identifying the nucleic acid hypermethylation state of one or more genes comprising gene specific primers for use in polymerase chain reaction (PCR), and instructions for use. The invention also features kits for detecting cancer by detecting nucleic acid hypermethylation of one or more genes, the kit comprising gene specific primers for use in polymerase chain reaction (PCR), and instructions for use. As described above, the PCR, in particularly preferred examples, is quantitative methylation specific PCR (QMSP). In some embodiments, QMSP is combined with methylation on beads (MOB).

In certain embodiments, any gene comprising one or more CpG islands in the promoter region can be detected using the kits of the invention. In certain preferred examples, the one or more genes are selected from the group consisting of genes involved in tumor suppression, nucleic acid repair, anti-proliferation, apoptosis, ras signaling, adhesion, differentiation, development, and cell cycle regulation.

In certain preferred embodiments of the invention the genes can be detected in a panel consisting of the following:
(1) genes involved in tumor suppression and cell adhesion
(2) genes involved in cell cycle regulation and adhesion
(3) genes involved in tumor suppression and cell cycle regulation
(4) genes involved in ras signaling and cell cycle control

In certain examples, the genes are selected from the group consisting of: CDO1, SOOX17, HOXA7, HOXA9, TAC1, ZFP42, and combinations thereof.

The kits can be used to detect hypermethylation of at least one of the genes as described herein. In some examples, the techniques herein can be used to detect hypermethylation of one or more of the genes as described herein. The one or more genes can be selected from the following: CDO1, SOOX17, HOXA7, HOXA9, TAC1, ZFP42, and combinations thereof.

The invention also provides kits for the diagnosis or monitoring of a neoplasia in a biological sample obtained from a subject. In various embodiments, the kit includes at least one primer or probe whose binding distinguishes between a methylated and an unmethylated sequence, together with instructions for using the primer or probe to identify a neoplasia. In another embodiment, the kit further comprises a pair of primers suitable for use in a polymerase chain reaction (PCR). In yet another embodiment, the kit further comprises a detectable probe. In yet another embodiment, the kit further comprises a pair of primers capable of binding to and amplifying a reference sequence. In yet other embodiments, the kit comprises a sterile container which contains the primer or probe; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Cancer Screening

Ideally, methods for cancer screening should be easy to perform, cost-effective, noninvasive, and provide a benefit to patients. Current methods of screening for lung cancer are inadequate. For example, lung cancer is mainly diagnosed at an advanced local or metastatic stage in almost 67% of cases.^{2,3} This tendency to diagnose lung cancer at a late stage results in poor survival with 16.8% probability of survival at five-years from the time of diagnosis.² The present invention provides significant advantages over existing screening technologies for lung and other cancers. Significantly, the invention provides methods for detecting methylation of genes including, but not limited to, CDO1, SOOX17, HOXA7, HOXA9, TAC1, and ZFP42. Increased methylation in CDO1, SOOX17, HOXA7, HOXA9, TAC1, and ZFP42 is detected at all stages of lung cancer.

Methylation-on-Beads is a single-tube method for polynucleotide extraction and bisulfite conversion that provides a rapid and highly efficient method for DNA extraction, bisulfite treatment and detection of DNA methylation using silica superparamagnetic particles (SSP). All steps are implemented without centrifugation or air drying that provides superior yields relative to conventional methods for DNA extraction and bisulfite conversion. SSP serve as solid substrate for DNA binding throughout the multiple stages of each process. Specifically, SSP are first used to capture genomic DNA from raw tissue samples, processed tissue samples or cultured cells. Sodium bisulfite treatment is then carried out in the presence of SSP without tube transfers. Finally, the bisulfite treated DNA is analyzed to determine the methylation status. DNA extraction yield was found to be 35-55 times the yield from conventional extraction. 90% of the input DNA was recovered after bisulfite treatment. In addition, Methylation-on-Beads total process time was completed in less than 6 hours when compared to 3 days for conventional methods. Hence, Methylation-on-Beads allows for convenient, efficient and contamination-resistant methylation detection in a single tube or other reaction platform. Methods for carrying out methylation-on-beads are known in the art, and described, for example, in PCT/US2009/000039, which is incorporated herein in its entirety.

### Types of Biological Samples

The level of promoter methylation in each of the genes identified herein (e.g., CDO1, SOOX17, HOXA7, HOXA9, TAC1, and ZFP42) can be measured in different types of biologic samples. In one embodiment, the biologic sample is a blood, plasma, or serum sample. In another embodiment, the sample is saliva, sputum, mucous, or other bodily fluids. In another embodiment, the biologic sample is a biologic fluid sample (e.g., blood, blood serum, plasma, urine, lung lavage, tears, mucous, sweat or any other biological fluid useful in the methods of the invention).

### Diagnostic Assays

The present invention provides a number of diagnostic assays that are useful for the identification or characterization of a neoplasia (e.g., lung cancer). In one embodiment, a neoplasia is characterized by quantifying or determining the methylation level of one or more, but not limited to, of the following promoters: CDO1, SOOX17, HOXA7, HOXA9, TAC1, and ZFP42 in the neoplasia. In one embodiment, methylation levels are determined using quantitative methylation specific PCR (QMSP) to detect CpG methylation in genomic DNA. QMSP uses sodium bisulfate to convert unmethylated cytosine to uracil. A comparison of sodium bisulfate treated and untreated DNA provides for the detection of methylated cytosines.

While the examples provided below describe methods of detecting methylation levels using QMSP, the skilled artisan appreciates that the invention is not limited to such methods. Methylation levels are quantifiable by any standard method, such methods include, but are not limited to real-time PCR, Southern blot, bisulfite genomic DNA sequencing, restriction enzyme-PCR, MSP (methylation-specific PCR), methylation-sensitive single nucleotide primer extension (MS-SNuPE) (see, for example, Kuppuswamy et al., Proc. Natl Acad. Sci. USA, 88, 1143-1147, 1991), DNA microarray based on fluorescence or isotope labeling (see, for example, Adorján Nucleic Acids Res., 30: e21 and Hou Clin. Biochem., 36:197-202, 2003), mass spectroscopy, methyl accepting capacity assays, and methylation specific antibody binding. See also U.S. Pat. Nos. 5,786,146; 6,017,704; 6,300,756; and 6,265,171.

The primers used in the invention for amplification of the CpG-containing nucleic acid in the specimen, after bisulfite modification, specifically distinguish between untreated or unmodified DNA, methylated, and non-methylated DNA. Methylation specific primers for the non-methylated DNA preferably have a T in the 3′ CG pair to distinguish it from the C retained in methylated DNA, and the compliment is designed for the antisense primer. Methylation specific primers usually contain relatively few Cs or Gs in the sequence since the Cs will be absent in the sense primer and the Gs absent in the antisense primer (C becomes modified to U(uracil) which is amplified as T(thymidine) in the amplification product).

The primers of the invention embrace oligonucleotides of sufficient length and appropriate sequence so as to provide specific initiation of polymerization on a significant number of nucleic acids in the polymorphic locus. Specifically, the term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides. Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting methylated nucleic acid.

In one embodiment, methylation specific primers amplify a desired genomic target using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of a methylation specific PCR product.

The methylation level of genes including, but not limited to, CDO1, SOOX17, HOXA7, HOXA9, TAC1, and ZFP42 promoters described herein defines the methylation profile of a neoplasia (e.g., lung cancer). The level of methylation present at any particular promoter is compared to a reference. In one embodiment, the reference is the level of methylation present in a control sample obtained from a patient that does not have a neoplasia. In another embodiment, the reference is a baseline level of methylation present in a biologic sample derived from a patient prior to, during, or after treatment for a neoplasia. In yet another embodiment, the reference is a standardized curve.

The methylation level of any one or more of the promoters described herein (e.g., CDO1, SOOX17, HOXA7, HOXA9, TAC1, and ZFP42) is used, alone or in combination with other standard methods, to determine the stage or grade of a neoplasia. Grading is used to describe how abnormal or aggressive the neoplastic cells appear, while staging is used to describe the extent of the neoplasia. The grade and stage of the neoplasia is indicative of the patient's long-term prognosis (i.e., probable response to treatment and survival). Thus, the methods of the invention are useful for predicting a patient's prognosis, and for selecting a course of treatment.

In conventional diagnostic methods, a pathologist will view a tissue sample from the tumor and determine the grade based on the degree of pathology observed. High-grade neoplasias are the most deadly because they are most aggressive and fast growing. High-grade neoplasias typically move rapidly into surrounding tissues, such as lymph nodes and bones.

Stage refers to the extent of a cancer. In lung cancer, a tumor up to 5 cm wide that has not spread to any lymph nodes or other organs is classified as stage I. These tumors are usually resectable (able to be removed surgically). Stage IA NSCLC lung cancer tumors are characterized by a tumor 3 cm or smaller. For Stage IB lung cancer, a tumor is 3-5 cm wide in any direction. Stage II tumors are a little larger than stage I, may have spread to lymph nodes on the same side of the chest, and/or may have begun to invade other structures within the chest. These tumors are usually resectable. In Stage IIA lung cancer, a tumor 5-7 cm wide in any direction with no spread to lymph nodes or less than 5 cm, but spread to lymph nodes on the same side of the chest. In Stage IIB lung cancer, tumors are 7 cm or wider in any direction with no spread to lymph nodes or are 5-7 cm wide, but spread to lymph nodes on the same side of the chest or are beginning to invade structures within the chest or there is more than one tumor in the same lobe of the lung. A tumor that has spread to lymph nodes beyond the same side of the chest, but does not appear to have spread to other organs outside the chest is classified as stage III. Often, stage III tumors are unresectable (unable to be removed surgically). In Stage IIIA a tumor has spread to lymph nodes in the center of the chest. In Stage IIIB a tumor spread to lymph nodes on the opposite side of the chest or involves major structures, such as the heart or arteries. Stage IV lung cancer is accompanied by pleural effusion (a fluid build-up between the lungs and the chest wall) or that has metastasized (spread) to other parts of the body.

### Selection of a Treatment Method

Identifying the presence of increased promoter methylation in genes including, but not limited to, CDO1, SOX17, HOXA7, HOXA9, TAC1, and ZFP42, indicates that the subject likely is at risk of developing lung cancer. Typically, after the subject has been identified as having increased promoter methylation in one or more of the CDO1, SOX17, HOXA7, HOXA9, TAC1, and ZFP42 genes, imaging studies are carried out. Such studies include, but are not limited to, endoscopic ultrasound, MRI, CT scan, PET scan. After the location of the cancer (e.g., lung cancer), a method of treatment is selected. More aggressive neoplasias are less susceptible to conservative treatment methods. When methods of the invention indicate that a neoplasia is very aggressive (e.g., lung cancer), an aggressive method of treatment should be selected. Aggressive therapeutic regimens typically include one or more of the following therapies: surgical resection, radiation therapy, and chemotherapy.

### Patient Monitoring

The diagnostic methods of the invention are also useful for monitoring the course of a lesion in a patient or for assessing the efficacy of a therapeutic regimen. In one embodiment, the diagnostic methods of the invention are used periodically to monitor the methylation levels of genes including, but not limited to, CDO1, SOX17, HOXA7, HOXA9, TAC1, or ZFP42 and combinations thereof. In one example, the neoplasia is characterized using a diagnostic assay of the invention prior to administering therapy. This assay provides a baseline that describes the methylation level of one or more promoters or the methylation profile of the neoplasia prior to treatment. Additional diagnostic assays are administered during the course of therapy to monitor the efficacy of a selected therapeutic regimen. A therapy is identified as efficacious when a diagnostic assay of the invention detects a decrease in methylation levels at one or more promoters relative to the baseline level of methylation.

### Microarray Procedure

The methods of the invention may also be used for microarray-based assays that provide for the high-throughput analysis of methylation at a large numbers of genes and CpG dinucleotides in parallel. Such methods are known in the art, and are described, for example, in U.S. Pat. No. 6,214,556. (See also, Adorjan et al., Nucleic Acids Research, 30:e21, 2002). In brief, oligonucleotides with a C6-amino modification at the 5′-end are immobilized on a solid substrate at fixed positions to form an array. Useful substrate materials include membranes, composed of paper, nylon or other materials, filters, chips, glass slides, and other solid supports. The ordered arrangement of the array elements allows hybridization patterns and intensities to be interpreted as methylation levels of particular genes. For each analyzed CpG position two oligonucleotides, reflecting the methylated and non-methylated status of the CpG dinucleotides, are immobilized at specific loci on the array. Oligonucleotides may be designed to match only the bisulphite-modified DNA fragments; this excludes signals arising from incomplete bisulphite conversion. The oligonucleotide microarrays are hybridized with detectably labeled PCR products. Such PCR products are amplified from a biological sample using any method known in the art. Hybridization conditions are optimized to allow detection of the differences between the TG and CG variants. Exemplary hybridization conditions are described herein. Subsequently, images of the hybridized arrays are obtained using any desired detection method. The degree of methylation at any specific CpG position can then be quantified.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

It should be appreciated that the invention should not be construed to be limited to the examples that are now described; rather, the invention should be construed to include any and all applications provided herein and all equivalent variations within the skill of the ordinary artisan.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

The present experiments examine the DNA methylation of the CDO1, TAC1, HOXA7, HOXA9, SOX17 and ZFP42 genes in lung cancer patients. The results show that methylation of one more genes including, but not limited to, CDO1, TAC1, HOXA7, HOXA9, SOX17 and ZFP42 is a predictive biomarker for risk of developing lung cancer and is detected in early stages from blood/plasma and sputum samples.

### Example 1: Patients and Methods

The National Lung Screening Trial (NLST) showed a 20% reduction in lung cancer mortality using low-dose computed tomography (CT) screening, but with a 96.4% false positive rate. Lung cancer screening can be improved through cancer-specific biomarkers using an in-vitro assay to detect cancer DNA in body fluids. Improvements to the diagnostic accuracy of lung cancer diagnosis using gene promoter methylation in sputum and plasma through the use of Methylation On Beads (MOB) and a highly specific panel of genes for detection of lung cancer was sought.

A retrospective case-control study involving subjects who had nodules suspicious for lung cancer on CT imaging, and who subsequently underwent surgery was performed. Plasma and sputum were obtained pre-operatively. Cases had pathological confirmation of non-small cell lung cancer (NSCLC) stage IA, IB and IIA while controls were pathologically free of cancer. Promoter hypermethylation levels and the amplification cycle threshold were quantified by using MOB and quantitative methylation specific real-time PCR in cancer-specific genes that were previously identified from the Cancer Genome Atlas (TCGA). This panel of genes included: CDO1, TAC1, HOXA7, HOXA9, SOX17 and ZFP42.

### Study Population

From a prospective observational study of 651 participants, initiated in 2007 under the auspices of the Lung Cancer Specialized Program of Research Excellence (SPORE), to monitor cancer recurrence after surgery in early stage NSCLC patients (T1-T2 NO), a total of 210 study patients were obtained. Excluded patients included 84 with lung cancer lesions other than stages IA, IB and IIA were 9 with positive surgical margins, 126 with positive lymph node infiltration or metastasis, 23 due to metastatic disease from other organs and 199 with absence of any sample available. Institutional review board (IRB) approval was obtained prior to the start of this study (NA_00005998), and all patients signed informed consent. Surgical resection with curative intent and pathological analyses for suspected lung cancer lesions were completed in all patients. Patients were staged according to the new revised TNM guidelines classification criteria.²⁹ Cases were defined as patients with confirmed lung cancer lesion by pathology. Controls were defined as patients confirmed to have non-cancerous lesions in their surgical specimens. Plasma and sputum samples were obtained on all patients prior to surgical resection. The information collected from clinical notes included demographics, co-morbidities, pack-year, smoking Status, pulmonary function tests. Former smokers were defined as those individuals who had quit smoking within 15 year at the time of study. Pack-years of cigarette smoking was defined as the average number of packs smoked per day multiplied by the number of years of smoking. Nodule size was obtained from the pathological report. Nodule volume obtained from surgical pathological reports was calculated with the ellipsoid volume formula (Volume=4/3×π×radius A×radius B×radius C).

### Plasma and Sputum Collection

Prior to surgery, 2 ml of plasma sample was collected in tubes containing sodium heparin (Bectin Dickinson, Franklin Lakes) and then stored at −80° C.

For sputum collection two cups containing Saccomannos' fixative solution were used for each patient as previously described.^{15,18,30} Subjects were asked to provide an early morning spontaneous sputum at home in two cups for 3 consecutive days within 1 week prior to pulmonary resection.^{18,31} Five ml of sputum was collected, washed with Saccomanos' solution, vortexed, centrifuged and then stored at −80° C.¹⁵

### DNA Isolation and Bisulfite Conversion

DNA extraction from plasma and sputum was performed by using MOB process that allows DNA extraction and bisulfite conversion in a single tube via the use of silica super magnetic beads.²⁵ This approach yields a 1.5 to 5-fold improvement in extraction efficiency with small amount of DNA in comparison to traditional conventional techniques.²⁷ The protocol previously described was optimized for plasma²⁷, using 1.5 ml of plasma and 375 ul (800 units/ml, NEBL p8107s) of proteinase K.

For DNA extraction from sputum using the MOB method, the protocol was modified to use with plasma by adding 200 μl of sample to 300 μl of Buffer AL and 40 μl of Proteinase K and by incubating together at the same temperature (50° C. for 2 hours). After digestion, 300 μl of IPA and 150 μl of beads were added. The lysate was also incubated and rotated for 10 minutes before adding 5 μl of carrier RNA and incubating for an additional 5 minutes.²⁷

### DNA Methylation Analysis

The genomic sequence for the genes and 1000 bases upstream were obtained from the UCSC genomic browser website.³² Primers and probes for methylation analysis were designed by using primer3 (v.0.4.0).^{33,34} All primer sequences are listed in supplementary Table 11. Quantification Methylation-Specific PCR (QMSP) was performed as previously described with some modification.⁷

TABLE 11
Methylation Specific PCR primers and probes

| | | | | Fragment | Annealing |
|---|---|---|---|---|---|
| Gene | Forward 5′-3′ | Reverse 5′-3′ | Probe 5′ | Size (bp) | Temp. |
| CDO1 | AGGCGGGGAGAT | CCTAAAACGCCGAA | CGGTTTACGCGTATATTTTCGGTTT | 97 | 65 |
| | TTTGCG | AACAACG | T | | |
| TAC1 | CGCGTGGGGAGA | TAATCGCTCCGCACT | CGTAAGGTATTGAGTAG | 132 | 65 |
| | ATGTTACG | CTCG | GCGAAAGAGCGCGTTCG | | |
| HOXA9 | TTATTGTTTTAGAAGTT | TAAATTTTTACAACT | CGGAAACGATTAATAGATTCGTTT | 92 | 65 |
| | ATATAGGTTGGCG | AAAAACCACG | GTTTCG | | |
| HOXA7 | AGTAGTTTTTATAGGT | AACGCGAACGAATT | CGTAGTCGTTTAGAATGGAAGGGT | 98 | 65 |
| | GGTTTCGTTTCG | CTTTAACCG | AAGAGGt | | |
| SOX17 | TTTAACGACGCGGGAT | CCCAACCGACCTAAT | CGTTTTCGTCGTTTTATTGGTTATA | 105 | 65 |
| | CG | AACACTACG | TTTGTGTAG | | |
| ZFP42 | TTCGGGTTGAGGGTGA | CGACCCCGCCCTAA | CGTCGTTTAGGTGTTAGGCGGTTT | 103 | 65 |
| | GCG | AACG | CG | | |
| B-Actin | TAG GGA GTA TAT | AACACACAATAACA | CGACTGCGTGTGGGG | 103 | 65 |
| | AGG TTG GGG AAGTT | A ACA CAA ATT CAC | TGGTGATGGAGGAGG | | |
| | | | TTTAGGCAGTCG | | |

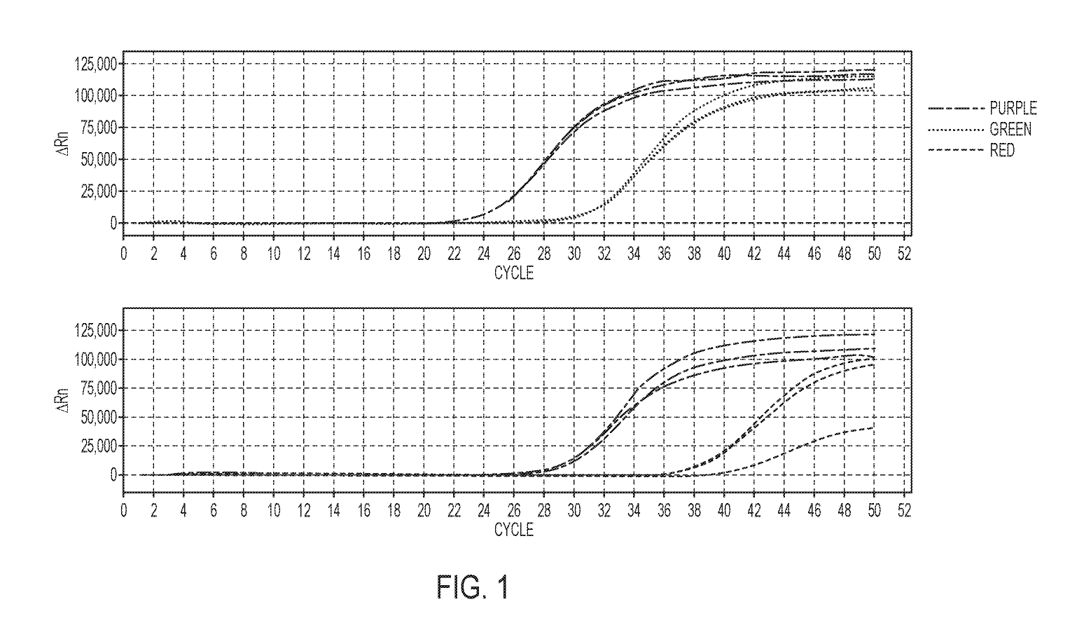

Two microliters of bisulfite converted DNA target was added to 25 μl of quantitative PCR reaction mixture that contained 2 μl of bisulfite converted DNA, 300 nM R-sense primer, 300 nM F-anti-sense primer, 100 nM probe, 100 nM of fluorescein reference dye (Life Technologies), 1.67 mM dNTPs (VWRQuotation), 1 μl of Platinum Taq® DNA Polymerase (invitrogen). Master mix contained 16.6 mM (NH4)2SO4, 67 mM Tris pH 8.8, 6.7 mM MgCl2 and 10 mM β-mercaptoethanol. Amplification reactions were carried using a 96 well-plates (MicroAmp®) patients DNA controls and water samples were analyzed on triplicates. Thermo cycling conditions was as follows: 95° C. for 5 min, 50 cycles at 95° C. for 15 seconds, and primer annealing 65° C. for 1 min, and 72° C. for 1 min, respectively. An ABI StepOne-PlusReal-Time PCR system was used (Applied Bio systems). All samples were determined positive if at least one of the three replicates demonstrated amplification and if the Cycle threshold (Ct) value for the target gene was ≤42 and the B-actin was less ≤35. qPCR curves were reviewed blinded by 3 independent investigators to avoid bias, and all irregular amplification curves were removed. For quantification analysis, a ΔCT value of 100 was assigned to samples that did not amplify. Cycle threshold (Ct) values for each gene (target) were transformed with the conversion expressed between parenthesis (Converted ΔCT=2^{-ΔCT}) and subsequently normalized for DNA input by calculating the ΔCT=Converted CT B-Actin (ACTB)−Converted CT (Target) (FIG. 1).

### Statistical Analysis

Quantitative data are expressed as median (interquartile range) for continuous, non-parametric variables and frequency (percentage) for categorical variables. For inter-group comparison, the Wilcoxon rank sum test was used for continuous data and the Fisher's exact test for categorical data. Receiver operator classification (ROC) curves and areas under the curve were obtained for the each one of the methylated genes and for the combination of the 3 genes with the largest area under the curve to assess its diagnostic accuracy for lung cancer. Sensitivity and specificity values were obtained from the optimum cutoff thresholds derived from the ROC curves (R statistic software, version 3.0.2, Vienna, Austria).³⁵ P-value of <0.05 was considered statistically significant. Areas under the curve were reported with 95% CIs.

Different gene combinations were analyzed using nonparametric machine learning approach using the normalized Ct methylation values from all genes obtained from plasma samples together with age, pack-year, COPD status and FVC values on one side and another model for all genes obtained from sputum on the other side. In particular, random forest was used to generate multiple classification models and final prediction was made using majority vote. The prediction models were developed with a training subset, in a random selection of ⅔ of the original study sample maintaining constant the proportion of cases and controls among subsets. Predictions for lung cancer risk were obtained for the testing subset (⅓ of the original sample) using the model obtained from the training subset. The complete data set (Training+Testing) contains 210 patients with 150 (71.4%) patients with lung cancer and 60 (28.6%) controls. The training dataset (⅔ of the complete dataset) has 140 patients with 99 (70.7%) cancer patients and 41 (29.3%) controls. The testing dataset (⅓ of the complete dataset) has 70 patients with 51 (72.9%) lung cancer patients and 19 (27.1%) controls. A statistician, who was responsible for these analyses, was blinded to the testing subset cancer status having limited access to only de-identified data. Cross-validation of the lung cancer prediction accuracy was assessed with sensitivity, specificity and areas under the curve.

### Example 2: Results

Of 651 patients observed, 150 subjects had T-T2N0 NSCLC and 60 patients had non-cancerous lesions. Six genes were methylated in significantly more people with cancer than non-cancer in both plasma and sputum (p<0.001) with the exception of HOXA9 in sputum. Sensitivity and specificity for lung cancer diagnosis from a three-gene combination for sputum was 93% and 79% and for plasma was 91% and 64%. The area under the Receiver Operating Curve for the three genes in sputum was 0.89 95% CI (0.80-0.98) and for the genes in plasma was 0.77 95% confidence interval (CI) (0.68-0.86).

### Characteristics of the Patients

A total of 210 patients fulfilled inclusion criteria with 150 subjects having lung cancer and 60 individuals with non-cancerous lung lesions (Table 1). Clinical and demographic variables were similar in cases and controls with the exception of age, number of pack-year and nodule size (cm) as well as volume (cm3). Subjects with lung cancer were significantly older than controls (p=0.007), smoked significantly more in pack-years (p=0.01), had significantly larger nodules (p=0.01) and had a significant larger proportion of nodules ≥2 cm (p=0.001). The proportion of smokers, former smokers and never smokers was identical between cases and controls. There was a distribution trend for African-Americans more likely to be cases than controls. Caucasians were equally distributed between cases and controls.

TABLE 1
Baseline Characteristics of the 210 Subjects

| Patient | Cancer | | Control | | |
|---|---|---|---|---|---|
| Characteristics | (N = 150) | | (N = 60) | | p Value |
| Age at surgery | 68 | (62-75) | 63 | (55-73) | 0.007 |
| (years) (IQR) | | | | | |
| Gender | | | | | |
| Male (%) | 63 | (42%) | 33 | (55%) | 0.094 |
| Female (%) | 87 | (58%) | 27 | (45%) | |
| Race | | | | | |
| White (%) | 120 | (80%) | 51 | (85%) | |
| Black (%) | 19 | (13%) | 3 | (5%) | 0.087 |
| Other (%) | 11 | (7%) | 6 | (10%) | |
| Stage | | | | | |
| IA-IB (%) | 136 | (91%) | NA | | |
| IIA (%) | 14 | (9%) | NA | | |
| Histology | | | | | |
| Adenocarcinoma (%) | 121 | (81%) | NA | | |
| Squamous-cell (%) | 26 | (17%) | NA | | NA |
| Adenosquamous (%) | 3 | (2%) | NA | | |
| Smoking status | | | | | |
| Current (%) | 27 | (18%) | 7 | (12%) | |
| Former (%) | 87 | (58%) | 34 | (57%) | 0.176 |
| Never (%) | 31 | (21%) | 19 | (32%) | |
| Pack-year (IQR) | 30 | (10-50) | 20 | (0-35) | 0.010 |
| COPD (%) | 41 | (27%) | 12 | (20%) | 0.370 |
| FEV1 % | 84 | (70-99) | 85 | (70-100) | 0.861 |
| Predicted (IQR) | | | | | |
| FVC % | 92 | (80-103) | 87 | (80-110) | 0.682 |
| Predicted (IQR) | | | | | |
| FEV1/FVC % | 73 | (68-78) | 77 | (70-79) | 0.080 |
| Ratio (IQR) | | | | | |
| Nodule size (cm) | 2 | (1.5-3) | 1.5 | (1.1-3) | 0.01 |
| <1 cm | 6 | (4%) | 13 | (22%) | |
| 1-2 cm | 52 | (35%) | 19 | (32%) | 0.001 |
| >2 cm | 92 | (61%) | 28 | (47%) | |
| Nodule volume (cm³) | 4.19 | (1.77-14-14) | 1.6 | (0.52-18.12) | 0.001 |

Abbreviations: Chronic obstructive pulmonary disease: COPD, Forced Expiratory Volume in one second: FEV1, Forced vital capacity: FVC, Interquartile range: IQR.
Nodule size % <1 cm, 1-2, >2 cm

### Distribution of DNA Methylation

A significantly higher percentage of patients with lung cancer had methylation in both plasma and sputum than those without cancer for all genes except for HOXA9 in sputum, which was methylated in over 50% of people in both cases and controls (Table 2). In both, plasma and sputum, HOXA9 was the gene that showed the highest percentage of positively methylation of controls with 48% of controls methylated in blood and 58% of controls methylated in sputum. In other words, HOXA9 tend to be ubiquitously methylated, note being useful to differentiate cancer status.

TABLE 2
Rate of Gene Methylation in Blood and Sputum

| | Cancer | Control | |
|---|---|---|---|
| Blood | (n = 125) | (n = 50) | p Value |
| CDO1 | 81 (65%) | 13 (26%) | <0.001 |
| TAC1 | 95 (76%) | 11 (22%) | <0.001 |
| HOXA7 | 41 (33%) | 3 (6%) | <0.001 |
| HOXA9 | 101 (81%) | 24 (48%) | <0.001 |
| SOX17 | 89 (71%) | 7 (14%) | <0.001 |
| ZPF42 | 101 (81%) | 21 (42%) | <0.001 |
| CDO1, TAC1, SOX17 | 114 (91%) | 18 (36%) | <0.001 |
| | Cancer | Control | |
| Sputum | (n = 90) | (n = 24) | p Value |
| CDO1 | 70 (78%) | 8 (33%) | <0.001 |
| TAC1 | 76 (84%) | 5 (21%) | <0.001 |
| HOXA7 | 57 (63%) | 2 (8%) | <0.001 |
| HOXA9 | 69 (77%) | 14 (58%) | 0.119 |
| SOX17 | 76 (84%) | (12%) | <0.001 |
| ZPF42 | 79 (88%) | 9 (38%) | <0.001 |
| TAC1, HOXA7, SOX17 | 84 (93%) | 5 (21%) | <0.001 |

p values were calculated with the use of the Fisher's exact test.

TABLE 3A
Gene Methylation Sensitivity, Specificity, AUC, and Association
with Cancer Diagnosis for genes obtained from Blood.

| | Sensi- | Speci- | | | | |
|---|---|---|---|---|---|---|
| Blood | tivity | ficity | PPV | NPV | AUC | 95% CI |
| CDO1 | 65% | 74% | 86% | 46% | 0.68 | (0.58-0.77) |
| TAC1 | 76% | 78% | 90% | 57% | 0.78 | (0.70-0.86) |
| HOXA7 | 33% | 94% | 93% | 36% | 0.60 | (0.51-0.69) |
| HOXA9 | 81% | 52% | 81% | 52% | 0.62 | (0.52-0.73) |
| SOX17 | 71% | 86% | 93% | 54% | 0.78 | (0.70-0.86) |
| ZFP42 | 81% | 58% | 83% | 55% | 0.66 | (0.56-0.75) |
| CD01, TAC1, | 91% | 64% | 86% | 74% | 0.77 | (0.68-0.86) |
| SOX17 | | | | | | |

Abbreviations: area under the curve (in the ROC curves): AUC, 95% confidence interval: 95% CI, Odds Ratio: OR.
* OR was adjusted by Age, Pack-years and Nodule size

TABLE 3B
Gene Methylation Sensitivity, Specificity, AUC, and Association
with Cancer Diagnosis for genes obtained from Sputum

| | Sensi- | Speci- | | | | |
|---|---|---|---|---|---|---|
| Sputum | tivity | ficity | PPV | NPV | AUC | 95% CI |
| CDO1 | 78% | 67% | 90% | 45% | 0.70 | (0.57-0.84) |
| TAC1 | 84% | 79% | 94% | 57% | 0.84 | (0.74-0.94) |
| HOXA7 | 63% | 92% | 97% | 40% | 0.77 | (0.67-0.86) |
| HOXA9 | 77% | 42% | 83% | 32% | 0.56 | (0.41-0.69) |
| SOX17 | 84% | 88% | 96% | 59% | 0.84 | (0.75-0.94) |
| ZFP42 | 88% | 62% | 90% | 58% | 0.73 | (0.60-0.87) |
| TAC1, HOXA7, | 93% | 79% | 94% | 75% | 0.89 | (0.80-0.98) |
| SOX17 | | | | | | |

Abbreviations: area under the curve (in the ROC curves): AUC, 95% confidence interval: 95% CI, Odds Ratio: OR.
* OR was adjusted by Age, Pack-years and Nodule size.

TABLE 4
Cross-validation of the lung cancer predictive
model in the testing subset

| | Sensi- | Speci- | | | | |
|---|---|---|---|---|---|---|
| | tivity | ficity | PPV | NPV | AUC | 95% CI |
| Prediction | 93% | 67% | 87% | 80% | 0.89 | 0.79-0.99 |
| from Blood | | | | | | |
| Prediction | 93% | 86% | 96% | 75% | 0.85 | 0.59-1 |
| from Sputum | | | | | | |

Abbreviations: area under the curve (in the ROC curves): AUC, 95% confidence interval: 95% CI.

The assay was also robust when only smokers were considered (n=155; 114 with cancer and 41 without cancer) (Tables 5-7 for Smokers Only). In never smokers, however, (n=40; 31 with lung cancer and 19 without lung cancer), almost all genes were significantly more methylated in the cancer group than in the non-cancer group with the exception of HOXA7 in plasma and HOXA9 in sputum.

TABLE 5
Baseline Characteristics of the 155 Subjects
(Former and current Smokers Only)

| Patient | Cancer | | Control | | |
|---|---|---|---|---|---|
| Characteristics | (N = 114) | | (N = 41) | | p Value |
| Age at surgery | 67 | (62-75) | 63 | (55-73.25) | 0.007 |
| (years) (IQR) | | | | | |
| Gender | | | | | |
| Male (%) | 48 | (42%) | 26 | (63%) | 0.03 |
| Female (%) | 66 | (58%) | 15 | (37%) | |
| Race | | | | | |
| White (%) | 94 | (83%) | 36 | (88%) | |
| Black (%) | 15 | (13%) | 2 | (5%) | 0.06 |
| Other (%) | 5 | (4%) | 3 | (7%) | |
| Stage | | | | | |
| IA-IB (%) | 103 | (90%) | NA | | NA |
| IIA (%) | 11 | (10%) | NA | | |
| Histology | | | | | |
| Adenocarcinoma (%) | 87 | (76%) | NA | | |
| Squamous-cell (%) | 24 | (21%) | NA | | NA |
| Adenosquamous (%) | 3 | (3%) | NA | | |
| Smoking status | | | | | |
| Current (%) | 27 | (24%) | 7 | (17%) | |
| Former (%) | 87 | (76%) | 34 | (83%) | 0.5 |
| Never (%) | 0 | (0%) | 0 | (0%) | |
| Pack-year (IQR) | 30 | (10-50) | 20 | (0-35) | 0.01 |
| COPD (%) | 34 | (32%) | 10 | (28%) | 0.83 |
| FEV1 % | 84 | (70-99) | 85 | (70-100) | 0.86 |
| Predicted (IQR) | | | | | |
| FVC % | 91 | (80-103) | 87 | (80-110) | 0.68 |
| Predicted (IQR) | | | | | |
| FEV1/FVC % | 73 | (68-77) | 77 | (70-80) | 0.07 |
| Ratio (IQR) | | | | | |
| Nodule size (cm) | 2 | (1.5-7.5) | 1.5 | (1-3) | 0.01 |
| <1 cm | 4 | (3%) | 12 | (30%) | |
| 1-2 cm | 37 | (33%) | 13 | (32%) | <0.001 |
| >2 cm | 73 | (64%) | 16 | (39%) | |
| Nodule volume (cm³) | 17.9 | (1.8-14.1) | 1.6 | (0.5-7.9) | <0.001 |

Abbreviations: Chronic obstructive pulmonary disease: COPD, Forced Expiratory Volume in one second: FEV1, Forced vital capacity: FVC, Interquartile range: IQR.
Nodule size % <1 cm, 1-2, >2 cm

TABLE 6
Rate of Gene Methylation in Blood and Sputum - Smokers Only

| | Cancer | Control | |
|---|---|---|---|
| Blood | (n = 96) | (n = 32) | p Value |
| CDO1 | 64 (67%) | 5 (16%) | <0.001 |
| TAC1 | 75 (78%) | 7 (22%) | <0.001 |
| HOXA7 | 33 (34%) | 3 (9%) | 0.006 |
| HOXA9 | 85 (89%) | 15 (47%) | <0.001 |
| SOX17 | 68 (71%) | 5 (16%) | <0.001 |
| ZPF42 | 79 (82%) | 13 (41%) | <0.001 |
| CDO1, TAC1, SOX17 | 89 (93%) | 9 (28%) | <0.001 |
| | Cancer | Control | |
| Sputum | (n = 67) | (n = 20) | p Value |
| CDO1 | 61 (91%) | 11 (55%) | <0.001 |
| TAC1 | 54 (81%) | 3 (15%) | <0.001 |
| HOXA7 | 45 (67%) | 3 (15%) | <0.001 |
| HOXA9 | 48 (72%) | 11 (55%) | 0.18 |
| SOX17 | 53 (79%) | 3 (15%) | <0.001 |
| ZPF42 | 54 (81%) | 6 (30%) | <0.001 |
| TAC1, SOX17, ZPF42 | 63 (94%) | 4 (20%) | <0.001 |

p values were calculated with the use of the Fisher's exact test.

TABLE 7A
Gene Methylation Sensitivity, Specificity, AUC and Association with
Cancer Diagnosis for genes obtained from Blood - Smokers Only.

| | Sensi- | Speci- | | | | |
|---|---|---|---|---|---|---|
| Blood | tivity | ficity | PPV | NPV | AUC | 95% CI |
| CDO1 | 67% | 84% | 93% | 46% | 0.76 | (0.66-0.86) |
| TAC1 | 78% | 78% | 91% | 54% | 0.80 | (0.70-0.90) |
| HOXA7 | 34% | 91% | 92% | 32% | 0.58 | (0.47-0.68) |
| HOXA9 | 89% | 53% | 85% | 61% | 0.66 | (0.53-0.79) |
| SOX17 | 71% | 84% | 93% | 49% | 0.78 | (0.68-0.88) |
| ZFP42 | 82% | 60% | 86% | 53% | 0.69 | (0.58-0.80) |
| CD01, TAC1, | 93% | 72% | 91% | 77% | 0.85 | (0.76-0.94) |
| SOX17 | | | | | | |

Abbreviations: Positive predictive value: PPV, Negative Predictive Value: NPV, area under the curve (in the ROC curves): AUC, 95% confidence interval: 95% CI.

TABLE 7B
Gene Methylation Sensitivity, Specificity, AUC
and Association with Cancer Diagnosis for genes
obtained from Sputum - Smokers Only.

| | Sensi- | Speci- | | | | |
|---|---|---|---|---|---|---|
| Sputum | tivity | ficity | PPV | NPV | AUC | 95% CI |
| CDO1 | 91% | 45% | 85% | 60% | 0.66 | (0.50-0.81) |
| TAC1 | 81% | 85% | 95% | 57% | 0.84 | (0.73-0.95) |
| HOXA7 | 67% | 85% | 94% | 44% | 0.76 | (0.65-0.88) |
| HOXA9 | 72% | 45% | 81% | 32% | 0.55 | (0.41-0.70) |
| SOX17 | 79% | 85% | 95% | 55% | 0.81 | (0.70-0.92) |
| ZFP42 | 81% | 70% | 90% | 52% | 0.76 | (0.62-0.90) |
| TAC1, SOX17, | 94% | 80% | 94% | 80% | 0.89 | (0.79-0.99) |
| HOXA7 | | | | | | |

Abbreviations: Positive predictive value: PPV, Negative Predictive Value: NPV, area under the curve (in the ROC curves): AUC, 95% confidence interval: 95% CI.

### Gene Methylation and Lung Cancer Diagnostic Accuracy

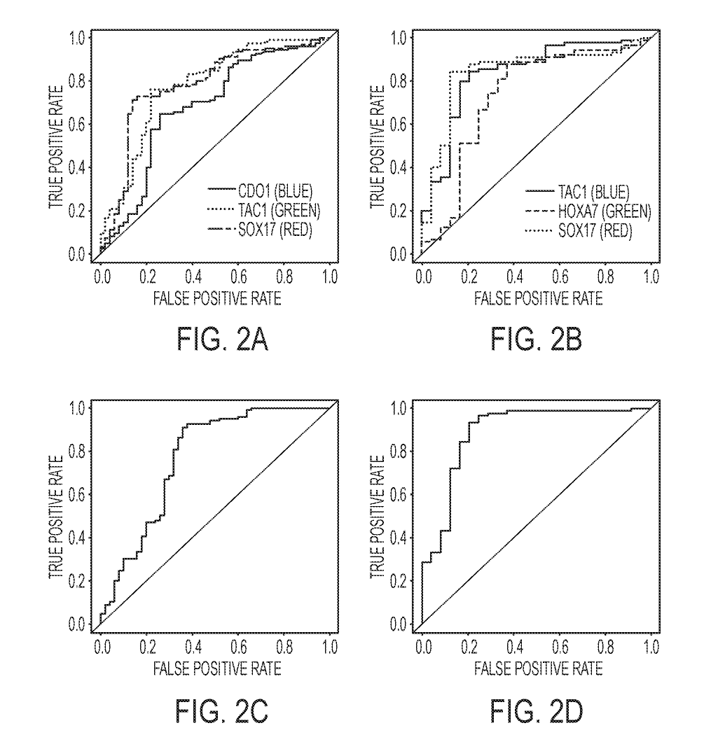

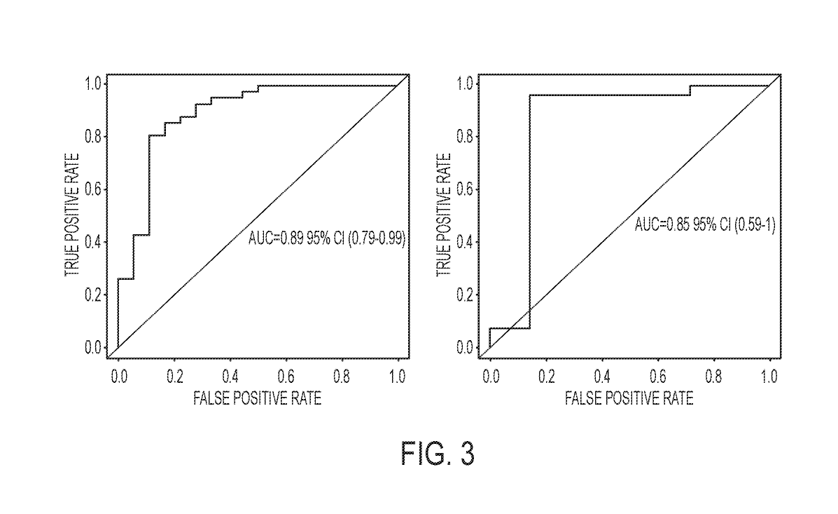

ROC curves for lung cancer detection were obtained for each single gene; using the normalized methylation Ct values (Tables 3A & 3B). The genes with the largest areas under the curve (AUC) were in sputum: TAC1 AUC: 0.84 95% CI (0.74-0.94), SOX17 AUC: 0.84 95% CI (0.75-0.94) and HOXA7 AUC: 0.77 95% CI (0.67-0.86) and in plasma: CDO1 AUC: 0.68 95% CI (0.58-0.77), TAC1 AUC: 0.78 (0.70-0.86) and SOX17 AUC: 0.78 95% CI (0.70-0.86) (FIG. 2). The sensitivity and specificity derived from the optimum cutoff point obtained from the ROC curve in the combination of TAC1, SOX17 and HOXA7 in sputum was 93% and 79% respectively and its corresponding ROC AUC was 0.89 95% CI (0.80-0.98). In plasma, the combination of CDO1, TAC1 and SOX17 showed a sensitivity, specificity and AUC of 91%, 64% and 0.77 95% CI (0.68-0.86) respectively (FIG. 3). HOXA9 in sputum had the lowest AUC value close to 0.5.

### Cross-Validation of Lung Cancer Risk Prediction

The complete data set (Training+Testing) contains 210 patients with 150 (71.4%) patients with lung cancer and 60 (28.6%) controls. The training dataset (⅔ of the complete dataset) has 140 patients with 99 (70.7%) cancer patients and 41 (29.3%) controls. The testing dataset (⅓ of the complete dataset) has 70 patients with 51 (72.9%) lung cancer patients and 19 (27.1%) controls.

When combining biomarkers using machine learning, the prediction accuracy when applied to independent validation data was 85% in plasma and 91% in sputum. The prediction obtained from sputum samples had a sensitivity, specificity and AUC of 93%, 86% and 0.85 95% CI (0.59-1) respectively and that the prediction obtained from blood samples showed a sensitivity, specificity and AUC of 93%, 67% and 0.89 95% CI (0.79-0.99) respectively (Table 4). Therefore, the application of the predictive models to the blinded testing subset was very similar to that obtained from the combination of the 3 genes with the largest area under the curve.

Similar screening and analyses were performed on samples from late stage lung cancer patients (see Tables 8, 9, 10).

TABLE 8
Baseline Characteristics of the 81 subjects
with stage lung cancer (III and IV)

| Patient | Cancer | | Control | | |
|---|---|---|---|---|---|
| Characteristics | (N = 21) | | (N = 60) | | p Value |
| Age at surgery | 67 | (62-75) | 63 | (55-73) | 0.007 |
| (years) (IQR) | | | | | |
| Gender | | | | | |
| Male (%) | 10 | (48%) | 33 | (55%) | 0.67 |
| Female (%) | 11 | (52%) | 27 | (45%) | |
| Race | | | | | |
| White (%) | 12 | (57%) | 51 | (85%) | |
| Black (%) | 6 | (29%) | 3 | (5%) | 0.01 |
| Other (%) | 3 | (14%) | 6 | (10%) | |
| Stage | | | | | |
| 3 (%) | 10 | (48%) | NA | | NA |
| 4 (%) | 11 | (52%) | NA | | |
| Histology | | | | | |
| Adenocarcinoma (%) | 11 | (52%) | NA | | |
| Squamous-cell (%) | 8 | (38%) | NA | | NA |
| Adenosquamous (%) | 2 | (10%) | NA | | |
| Smoking status | | | | | |
| Current (%) | 4 | (19%) | 7 | (12%) | |
| Former (%) | 15 | (71%) | 34 | (57%) | 0.12 |
| Never (%) | 2 | (10%) | 19 | (32%) | |
| Pack-year (IQR) | 30 | (10-50) | 19.5 | (0-35) | 0.01 |
| COPD (%) | 5 | (24%) | 12 | (22%) | 0.77 |
| FEV1 % | 84 | (70-99) | 85 | (70-100) | 0.86 |
| Predicted (IQR) | | | | | |
| FVC % | 91 | (80-103) | 87 | (80-110) | 0.68 |
| Predicted (IQR) | | | | | |
| FEV1/FVC % | 73 | (68-78) | 77 | (70-79) | 0.08 |
| Ratio (IQR) | | | | | |
| Nodule size (cm) | 2 | (1.5-3) | 1.5 | (1-3) | 0.01 |
| <1 cm | 2 | (9%) | 13 | (22%) | |
| 1-2 cm | 5 | (24%) | 19 | (32%) | 0.29 |
| >2 cm | 14 | (67%) | 28 | (47%) | |
| Nodule volume (cm³) | 4.2 | (1.8-14.1) | 1.6 | (0.5-7.9) | <0.001 |

Abbreviations: Chronic obstructive pulmonary disease: COPD, Forced Expiratory Volume in one second: FEV1, Forced vital capacity: FVC, Interquartile range: IQR.
Nodule size % <1 cm, 1-2, >2 cm

TABLE 9
Rate of Gene Methylation in Blood and
Sputum for Late Stage Cancer Subjects

| | Cancer | Control | |
|---|---|---|---|
| Blood | (n = 17) | (n = 50) | p Value |
| CDO1 | 12 (71%) | 13 (26%) | 0.003 |
| TAC1 | 9 (53%) | 7 (14%) | 0.002 |
| HOXA7 | 16 (94%) | 25 (50%) | 0.001 |
| HOXA9 | 12 (71%) | 22 (44%) | 0.09 |
| SOX17 | 16 (94%) | 30 (60%) | 0.01 |
| ZPF42 | 15 (88%) | 23 (46%) | 0.004 |
| CDO1, TAC1, SOX17 | 11 (65%) | 10 (20%) | 0.002 |
| | Cancer | Control | |
| Sputum | (n = 12) | (n = 24) | p Value |
| CDO1 | 10 (83%) | 8 (33%) | 0.01 |
| TAC1 | 10 (83%) | 4 (17%) | <0.001 |
| HOXA7 | 8 (67%) | 2 (8%) | <0.001 |
| HOXA9 | 10 (83%) | 13 (54%) | 0.14 |
| SOX17 | 12 (100%) | 3 (13%) | <0.001 |
| ZPF42 | 11 (92%) | 9 (38%) | 0.004 |
| TAC1, SOX17, HOXA7 | 12 (100%) | 4 (17%) | <0.001 |

p values were calculated with the use of the Fisher's exact test.

TABLE 10A
Gene Methylation Sensitivity, Specificity, AUC
and Association with Cancer Diagnosis for genes
obtained from Blood - Late Stage Subjects

| | Sensi- | Speci- | | | | |
|---|---|---|---|---|---|---|
| Blood | tivity | ficity | PPV | NPV | AUC | 95% CI |
| CDO1 | 71% | 74% | 48% | 88% | 0.75 | (0.62-0.88) |
| TAC1 | 53% | 86% | 56% | 84% | 0.73 | (0.59-0.86) |
| HOXA7 | 94% | 50% | 39% | 96% | 0.71 | (0.56-0.85) |
| HOXA9 | 71% | 56% | 35% | 85% | 0.55 | (0.40-0.70) |
| SOX17 | 94% | 40% | 35% | 95% | 0.63 | (0.48-0.78) |
| ZFP42 | 88% | 54% | 39% | 93% | 0.66 | (0.53-0.79) |
| CD01, TAC1, | 65% | 80% | 52% | 87% | 0.75 | (0.61-0.89) |
| SOX17 | | | | | | |

Abbreviations: Positive predictive value: PPV, Negative Predictive Value: NPV, area under the curve (in the ROC curves): AUC, 95% confidence interval: 95% CI.

TABLE 10B
Gene Methylation Sensitivity, Specificity, AUC
and Association with Cancer Diagnosis for genes
obtained from Sputum - Late Stage Subjects

| | Sensi- | Speci- | | | | |
|---|---|---|---|---|---|---|
| Sputum | tivity | ficity | PPV | NPV | AUC | 95% CI |
| CDO1 | 83% | 67% | 56% | 89% | 0.70 | (0.52-0.89) |
| TAC1 | 83% | 83% | 71% | 91% | 0.83 | (0.70-0.97) |
| HOXA7 | 67% | 92% | 80% | 85% | 0.72 | (0.49-0.94) |
| HOXA9 | 83% | 46% | 43% | 85% | 0.59 | (0.39-0.79) |
| SOX17 | 100% | 88% | 80% | 100% | 0.94 | (0.87-1.00) |
| ZFP42 | 92% | 63% | 55% | 94% | 0.71 | (0.54-0.88) |
| TAC1, SOX17, | 100% | 83% | 75% | 100% | 0.89 | (0.78-1.00) |
| HOXA7 | | | | | | |

Abbreviations: Positive predictive value: PPV, Negative Predictive Value: NPV, area under the curve (in the ROC curves): AUC, 95% confidence interval: 95% CI.

### Example 3: Discussion

According to the techniques herein, a sputum and plasma test that can identify patients with early stage NSCLC was developed. This assay has several characteristics which make it clinically applicable (i) it has a diagnostic sensitivity and specificity in sputum of 93% and 86%, respectively which achieves the diagnostic accuracy mandated by most clinical standards 10,37 (ii) it can be performed using minute quantities of sputum or serum (iii) it can distinguish CT detected malignant versus benign nodules making it applicable to ameliorating the current problem of high false positive screens (iv) this assay seems to be associated with a risk of having lung cancer independent of age, pack-year and nodule size.(v) it is able to diagnose early stage lung cancer in smokers making it likely to be applicable to asymptomatic, smokers at high risk for lung cancer (vi) the assay is relatively inexpensive, and PCR-based making it relatively simple to perform (vii) promoter methylation of the panel of genes involved,

The CDO1, TAC1, HOXA7, HOXA9, SOX17 and ZPF42 were previously identified in data from the Cancer Genome Atlas (TCGA) as being highly specific for lung cancer.^{23,28}

Previous studies have tried to achieve lung cancer risk assessment by the use of molecular biomarkers obtained from blood and sputum.^{15,17,18,30,31,38,39} However the achieved sensitivities and specificities were not high enough to be considered clinically applicable.^{15,17,18,30,31,38,39} One possible explanation could be that the former DNA extraction methods did not have enough efficiency for the small amount of DNA that can be harvested from samples such as sputum and blood. Instead, as previously demonstrated, Methylation-on-Beads (MOB) allows for increased throughput in DNA methylation detection and therefore an efficient and sensitive methylation detection.²⁵⁻²⁷

In addition, previous genes used for DNA methylation detection where primarily chosen from a candidate gene approach and were methylated in only a subset of lung tumors. The TCGA project identified methylation changes across different populations and tumor types. From the results of the lung cancer specific analyses, those genes with the highest methylation change in cancer were selected as candidates for a biomarker panel.²³

The biomarker data itself, including only the methylation level from the genes obtained from sputum samples, can accurately detect patients at risk of lung cancer better than the genes obtained from blood. On the other hand, the predictive model in which all genes from blood were considered simultaneously with age and number of pack-years showed a predictive accuracy closer to the one obtained from sputum. Non-smokers tend to struggle to produce quality sputa. In those cases in which sputum cannot be obtained, blood could be used as a secondary screening method for lung cancer detection.

According to the NLST results, the chances of having lung cancer with a positive CT screening are less than 5%.^{4,5} This is because lung cancer with CT screening in the NLST study yielded a 71% sensitivity and a 63% specificity with a 97% false positive rate.^{4,5} Our current findings indicate that the methylation detection in these panels of genes from blood and sputum could potentially be used in those patients that had positive results on CT screening to reduce false positive screening. Consequently these patients with positive CT screening and biomolecular profile of high risk of lung cancer could undergo further diagnostic testing including invasive procedures. Our findings support this possible change in management due to the fact that all our study subjects had lung lesions evident in CT scan that led to a surgical resection and posterior pathological confirmation.

On the other hand, according to the NLST data, CT screening can yield positive results in more than ⅓ of people.^{4,5} Therefore, an image first policy should shift towards a molecular profile first and an image confirmation later—reducing the number of patients that need to undergo further diagnostic invasive confirmation procedures.

The present invention shows it is possible to obtain high sensitivity and specificity for detection of lung cancer in early stages using a panel of methylated promoter genes in plasma and sputum, by using MOB and that the methylation level of this panel of genes is associated with lung cancer risk independent of age, pack-year and nodule size. These epigenetic biomarkers are used to identify patients with high risk of lung cancer development, reducing unnecessary tests and increasing the chance to diagnose lung cancer at earlier stages.

High diagnostic accuracy for early stage lung cancer is obtained using a panel of methylated promoter genes in sputum and plasma MOB. These epigenetic biomarkers are used to identify patients with high risk of lung cancer, thereby reducing false positive CT screening and the high costs of additional diagnostic tests.

### REFERENCES

1. Siegel R, Ma J, Zou Z, et al: Cancer statistics, 2014. CA Cancer J Clin 64:9-29, 2014.
2. Howlader N, Noone A, Krapcho M, et al: SEER Cancer Statistics Review, 1975-2011. National Cancer Institute. Bethesda, Md., USA, 2014.
3. Jett J R: Current treatment of unresectable lung cancer. Mayo Clin Proc 68:603-11, 1993.
4. National Lung Screening Trial Research Team, Aberle D R, Adams A M, et al: Reduced lung-cancer mortality with low-dose computed tomographic screening. N Engl J Med 365:395-409, 2011.
5. Tammemagi M C, Katki H A, Hocking W G, et al: Selection criteria for lung-cancer screening. N Engl J Med 368:728-36, 2013.
6. Bach P B, Mirkin J N, Oliver T K, et al: Benefits and harms of CT screening for lung cancer: a systematic review. JAMA 307:2418-29, 2012.
7. Herman J G, Graff J R, Myohanen S, et al: Methylation-specific PCR: a novel PCR assay for methylation status of CpG islands. Proc Natl Acad Sci USA 93:9821-6, 1996.
8. Belinsky S A, Nikula K J, Palmisano W A, et al: Aberrant methylation of p16(INK4a) is an early event in lung cancer and a potential biomarker for early diagnosis. Proceedings of the National Academy of Sciences of the United States of America 95:11891-11896, 1998.
9. Herman J G, Baylin S B: Gene silencing in cancer in association with promoter hypermethylation. N Engl J Med 349:2042-54, 2003.
10. Belinsky S A: Gene-promoter hypermethylation as a biomarker in lung cancer. Nat Rev Cancer 4:707-17, 2004.
11. Belinsky S A: Silencing of genes by promoter hypermethylation: key event in rodent and human lung cancer. Carcinogenesis 26:1481-7, 2005.
12. Baylin S B, Ohm J E: Epigenetic gene silencing in cancer—a mechanism for early oncogenic pathway addiction? Nat Rev Cancer 6:107-16, 2006.
13. Licchesi J D, Westra W H, Hooker C M, et al: Promoter hypermethylation of hallmark cancer genes in atypical adenomatous hyperplasia of the lung. Clin Cancer Res 14:2570-8, 2008.
14. Palmisano W A, Divine K K, Saccomanno G, et al: Predicting lung cancer by detecting aberrant promoter methylation in sputum. Cancer Res 60:5954-8, 2000.
15. Belinsky S A, Liechty K C, Gentry F D, et al: Promoter hypermethylation of multiple genes in sputum precedes lung cancer incidence in a high-risk cohort. Cancer Res 66:3338-44, 2006.
16. Brock M V, Hooker C M, Ota-Machida E, et al: DNA methylation markers and early recurrence in stage I lung cancer. The New England journal of medicine 358:1118-1128, 2008.
17. Ostrow K L, Hoque M O, Loyo M, et al: Molecular analysis of plasma DNA for the early detection of lung cancer by quantitative methylation-specific PCR. Clin Cancer Res 16:3463-72, 2010.
18. Leng S, Do K, Yingling C M, et al: Defining a gene promoter methylation signature in sputum for lung cancer risk assessment. Clin Cancer Res 18:3387-95, 2012.
19. Li L, Shen Y, Wang M, et al: Identification of the methylation of p14ARF promoter as a novel non-invasive biomarker for early detection of lung cancer. Clinical & translational oncology: official publication of the Federation of Spanish Oncology Societies and of the National Cancer Institute of Mexico 16:581-589, 2013.
20. Sandoval J, Mendez-Gonzalez J, Nadal E, et al: A prognostic DNA methylation signature for stage I non-small-cell lung cancer. J Clin Oncol 31:4140-7, 2013.
21. Kim Y, Kim D-H: CpG Island Hypermethylation as a Biomarker for the Early Detection of Lung Cancer, (null). New York, N.Y., Springer New York, 2014, pp 141-171.
22. Nawaz I, Qiu X, Wu H, et al: Development of a multiplex methylation specific PCR suitable for (early) detection of non-small cell lung cancer. Epigenetics 9:1138-1148, 2014.
23. Wrangle J, Machida E O, Danilova L, et al: Functional identification of cancer-specific methylation of CDO1, HOXA9, and TAC1 for the diagnosis of lung cancer. Clin Cancer Res 20:1856-64, 2014.
24. Yang X, Dai W, Kwong D L-w, et al: Epigenetic markers for noninvasive early detection of nasopharyngeal carcinoma by methylation-sensitive high resolution melting. International Journal of Cancer 136:E127-E135, 2014.
25. Bailey V J, Zhang Y, Keeley B P, et al: Single-tube analysis of DNA methylation with silica superparamagnetic beads. Clin Chem 56:1022-5, 2010.
26. Bailey V J, Keeley B P, Razavi C R, et al: DNA methylation detection using M S-qFRET, a quantum dot-based nanoassay. Methods 52:237-41, 2010.
27. Keeley B, Stark A, Pisanic T R, 2nd, et al: Extraction and processing of circulating DNA from large sample volumes using methylation on beads for the detection of rare epigenetic events. Clin Chim Acta 425:169-75, 2013.
28. Cancer Genome Atlas Research Network: Comprehensive genomic characterization of squamous cell lung cancers. Nature 489:519-25, 2012.
29. Ettinger D S, Wood D E, Akerley W, et al: NCCN Clinical Practice Guidelines in Oncology (NCCN Guidelines®) Non-small cell lung cancer, version 1.2015. J Natl Compr Canc Netw 12:1738-61, 2014.
30. Belinsky S A, Klinge D M, Dekker J D, et al: Gene promoter methylation in plasma and sputum increases with lung cancer risk. Clin Cancer Res 11:6505-11, 2005.
31. Prindiville S A, Byers T, Hirsch F R, et al: Sputum cytological atypia as a predictor of incident lung cancer in a cohort of heavy smokers with airflow obstruction. Cancer epidemiology, biomarkers & prevention: a publication of the American Association for Cancer Research, cosponsored by the American Society of Preventive Oncology 12:987-993, 2003.
32. Genome Bioinformatics Group of U C Santa Cruz: UCSC Genome Bioinformatics, 2015.
33. Brandes J C, Carraway H, Herman J G: Optimal primer design using the novel primer design program: MSPprimer provides accurate methylation analysis of the ATM promoter. Oncogene 26:6229-37, 2007.
34. Untergrasser A C I, Koressaar T, Ye J, Faircloth B C, Remm M, Rozen S G Primer3web, 2012.
35. Team R C: R: A language and environment for statistical computing, (ed R-Project Org, Version 3.0.2). Vienna, Austria, R Foundation for Statistical Computing, 2013.
36. Chen D C, Huang P, Cheng X Z: A concrete statistical realization of Kleinberg's stochastic discrimination for pattern recognition. part I. Two-class classification. Annals of Statistics 31:1393-1412, 2003.
37. Etzioni R, Urban N, Ramsey S, et al: The case for early detection. Nat Rev Cancer 3:243-52, 2003.
38. Kennedy T C, Proudfoot S P, Piantadosi S, et al: Efficacy of two sputum collection techniques in patients with air flow obstruction. Acta Cytol 43:630-6, 1999.
39. Kennedy T C, Proudfoot S P, Franklin W A, et al: Cytopathological analysis of sputum in patients with airflow obstruction and significant smoking histories. Cancer Res 56:4673-8, 1996.
40. Bianchi F, Nicassio F, Marzi M, et al: A serum circulating miRNA diagnostic test to identify asymptomatic high-risk individuals with early stage lung cancer. EMBO Mol Med 3:495-503, 2011.
41. Boeri M, Verri C, Conte D, et al: MicroRNA signatures in tissues and plasma predict development and prognosis of computed tomography detected lung cancer. Proc Natl Acad Sci USA 108:3713-8, 2011.
42. Hellman S. Stopping metastases at their source. N Engl J Med 1997; 337:996-7.
43. Riethmuller G, Johnson J P. Monoclonal antibodies in the detection and therapy of micrometastatic epithelial cancers. Curr Opin Immunol 1992; 4:647-55.
44. Zhu J J, Maruyama T, Jacoby L B, et al. Clonal analysis of a case of multiple meningiomas using multiple molecular genetic approaches: pathology case report. Neurosurgery 1999; 45:409-16.
45. Martini N, Bains M S, Burt M E, et al. Incidence of local recurrence and second primary tumors in resected stage I lung cancer. J Thorac Cardiovasc Surg 1995; 109:120-9.
46. Mountain C F. Revisions in the International System for Staging Lung Cancer. Chest 1997; 111:1710-7.
47. Hoffman P C, Mauer A M, Vokes E E. Lung cancer. Lancet 2000; 355:479-85.
48. Fearon, et al., Cell, 61:759, 1990.
49. Jones, et al., Cancer Res., 46:461, 1986.
50. Baylin, et al., Cancer Cells, 3:383, 1991.

1. A method of identifying a subject at risk of developing lung cancer comprising:
obtaining one or more samples from the subject;
extracting genomic DNA from the one or more samples;
performing a conversion reaction on the genomic DNA in vitro to convert unmethylated cytosine to uracil by deamination;
detecting nucleic acid methylation of one or more genes in the converted genomic DNA, wherein detecting nucleic acid methylation identifies a subject that is at risk of developing lung cancer.

2. The method of claim 1, wherein the sample is selected from the group consisting of blood, plasma, serum, saliva, sputum, and mucous.

3. The method of claim 1, wherein the detecting comprises a polymerase chain reaction (PCR) based technique.

4. The method of claim 3, wherein the PCR-based technique is selected from the group consisting of methylation on beads (MOB), quantitative methylation specific PCR (QMSP), multiplex-methylation specific PCR (MMSP), and combinations thereof.

5. The method of claim 1, wherein the nucleic acid methylation is in the promoter region of the one or more genes.

6. The method of claim 1, wherein the sample is blood or sputum.

7. The method of claim 1, further comprising:
determining a therapeutic regimen.

8. The method of claim 1, further comprising:
imaging the subject with one or more imaging modalities.

9. The method of claim 8, wherein the one or more imaging modalities are selected from the group comprising computed tomography (CT), ultrasound, magnetic resonance imaging (MRI), positron emission tomography (PET), optical imaging, and combinations thereof.

10. The method of claim 1, wherein the lung cancer is detected at an early stage.

11. The method of claim 1, wherein the method is performed prior to therapeutic intervention for cancer.

12. The method of claim 1, wherein the method is performed after therapeutic intervention for cancer.

13. The method of claim 1, wherein the subject has been diagnosed with cancer.

14. A method of treating a subject having or at risk of having cancer comprising:
obtaining one or more samples from the subject;
extracting genomic DNA from the one or more samples;
performing a conversion reaction on the genomic DNA in vitro to convert unmethylated cytosine to uracil by deamination;
detecting nucleic acid methylation of one or more genes in the converted genomic DNA, wherein the one or more genes are selected from the group consisting of CDO1, SOOX17, HOXA7, HOXA9, TAC1, and ZFP42, where presence of nucleic acid methylation indicates having or a risk of having lung cancer; and
administering to the subject a therapeutically effective amount of a chemotherapeutic agent, thereby treating a subject having or at risk for having cancer.

15-18. (canceled)

19. A kit for detecting cancer, comprising:
one or more reagents for extracting genomic DNA from the one or more samples;
one or more deamination reagents converting unmethylated cytosine in the extracted genomic DNA to uracil;
two or more primers for detecting nucleic acid methylation of one or more genes selected from the group consisting of CDO1, SOOX17, HOXA7, HOXA9, TAC1, and ZFP42; and,
instructions for use.

20. (canceled)

21. A method of identifying a subject at risk of developing lung cancer comprising:
obtaining one or more samples from the subject, wherein the sample is selected from the group consisting of blood, plasma, serum, saliva, sputum, and mucous;
extracting genomic DNA from the one or more samples;
performing a bisulfite conversion reaction on the genomic DNA in vitro to convert unmethylated cytosine to uracil by deamination;
amplifying, by a polymerase chain reaction (PCR) based technique, the bisulfite converted genomic DNA using one or more sets of gene specific primers to detect nucleic acid methylation of one or more corresponding genes in the converted genomic DNA, wherein detecting nucleic acid methylation identifies a subject that is at risk of developing lung cancer.

22. The method of claim 21, wherein amplifying step further comprises:
quantifying the amplified bisulfite converted DNA by monitoring hydrolysis of one or more molecular probes selected from the group consisting of a Taqman® probe and a Scorpion® probe.

23-29. (canceled)

30. A method of treating a subject having or at risk of having cancer comprising:
obtaining one or more samples from the subject;
extracting genomic DNA from the one or more samples;
performing a conversion reaction on the genomic DNA in vitro to convert unmethylated cytosine to uracil by deamination;
amplifying, by a polymerase chain reaction (PCR) based technique, the bisulfite converted genomic DNA using one or more sets of gene specific primers to detect nucleic acid methylation of one or more corresponding genes in the converted genomic DNA, wherein the one or more genes are selected from the group consisting of CDO1, SOOX17, HOXA7, HOXA9, TAC1, and ZFP42 and presence of nucleic acid methylation indicates having or a risk of having lung cancer; and
administering to the subject a therapeutically effective amount of a chemotherapeutic agent, thereby treating a subject having or at risk for having cancer.

31. The method of claim 30, wherein amplifying step further comprises:
quantifying the amplified bisulfite converted DNA by monitoring hydrolysis of one or more molecular probes selected from the group consisting of a Taqman® probe and a Scorpion® probe.

32. The method of claim 30, wherein the nucleic acid methylation of the one or more genes is compared to a threshold value that distinguishes between individuals with and without cancer.


SEQUENCE LISTING (21 sequences)

SEQ ID NO: 1 - DNA, 18 bp, Artificial Sequence
  Description of Artificial Sequence Synthetic primer
aggcggggagattttgcg

SEQ ID NO: 2 - DNA, 21 bp, Artificial Sequence
  Description of Artificial Sequence Synthetic primer
cctaaaacgccgaaaacaacg

SEQ ID NO: 3 - DNA, 26 bp, Artificial Sequence
  Description of Artificial Sequence Synthetic probe
cggtttacgcgtatattttcggtttt

SEQ ID NO: 4 - DNA, 20 bp, Artificial Sequence
  Description of Artificial Sequence Synthetic primer
cgcgtggggagaatgttacg

SEQ ID NO: 5 - DNA, 19 bp, Artificial Sequence
  Description of Artificial Sequence Synthetic primer
taatcgctccgcactctcg

SEQ ID NO: 6 - DNA, 34 bp, Artificial Sequence
  Description of Artificial Sequence Synthetic probe
cgtaaggtattgagtaggcgaaagagcgcgttcg

SEQ ID NO: 7 - DNA, 30 bp, Artificial Sequence
  Description of Artificial Sequence Synthetic primer
ttattgttttagaagttatataggttggcg

SEQ ID NO: 8 - DNA, 25 bp, Artificial Sequence
  Description of Artificial Sequence Synthetic primer
taaatttttacaactaaaaaccacg

SEQ ID NO: 9 - DNA, 30 bp, Artificial Sequence
  Description of Artificial Sequence Synthetic probe
cggaaacgattaatagattcgtttgtttcg

SEQ ID NO: 10 - DNA, 28 bp, Artificial Sequence
  Description of Artificial Sequence Synthetic primer
agtagtttttataggtggtttcgtttcg

SEQ ID NO: 11 - DNA, 23 bp, Artificial Sequence
  Description of Artificial Sequence Synthetic primer
aacgcgaacgaattctttaaccg

SEQ ID NO: 12 - DNA, 31 bp, Artificial Sequence
  Description of Artificial Sequence Synthetic probe
cgtagtcgtttagaatggaagggtaagaggt

SEQ ID NO: 13 - DNA, 18 bp, Artificial Sequence
  Description of Artificial Sequence Synthetic primer
tttaacgacgcgggatcg

SEQ ID NO: 14 - DNA, 24 bp, Artificial Sequence
  Description of Artificial Sequence Synthetic primer
cccaaccgacctaataacactacg

SEQ ID NO: 15 - DNA, 34 bp, Artificial Sequence
  Description of Artificial Sequence Synthetic probe
cgttttcgtcgttttattggttatatttgtgtag

SEQ ID NO: 16 - DNA, 19 bp, Artificial Sequence
  Description of Artificial Sequence Synthetic primer
ttcgggttgagggtgagcg

SEQ ID NO: 17 - DNA, 18 bp, Artificial Sequence
  Description of Artificial Sequence Synthetic primer
cgaccccgccctaaaacg

SEQ ID NO: 18 - DNA, 26 bp, Artificial Sequence
  Description of Artificial Sequence Synthetic probe
cgtcgtttaggtgttaggcggtttcg

SEQ ID NO: 19 - DNA, 26 bp, Artificial Sequence
  Description of Artificial Sequence Synthetic primer
tagggagtatataggttggggaagtt

SEQ ID NO: 20 - DNA, 27 bp, Artificial Sequence
  Description of Artificial Sequence Synthetic primer
aacacacaataacaaacacaaattcac

SEQ ID NO: 21 - DNA, 42 bp, Artificial Sequence
  Description of Artificial Sequence Synthetic probe
cgactgcgtgtggggtggtgatggaggaggtttaggcagtcg